(19) United States
(12) Patent Application Publication
(10) Pub. No.: US 2024/0132625 A1
(43) Pub. Date: Apr. 25, 2024

(54) A CONJUGATE, COMPOSITION AND METHOD FOR NONCOVALENT ANTIBODY CATENATION THAT INCREASES ANTIGEN-BINDING AVIDITY OF AN ANTIBODY IN PROPORTION TO THE DENSITY OF A TARGET ANTIGEN

(71) Applicant: KOREA ADVANCED INSTITUTE OF SCIENCE AND TECHNOLOGY, Daejeon (KR)

(72) Inventors: Byung-Ha OH, Daejeon (KR); Bo-Seong JEONG, Daejeon (KR); Seong-Woo KIM, Daejeon (KR); Seong-Bin IM, Daejeon (KR); Jinyeop SONG, Daejeon (KR)

(21) Appl. No.: 18/357,665

(22) Filed: Jul. 23, 2023

(30) Foreign Application Priority Data
Jul. 24, 2022 (KR) 10-2022-0091716
Jan. 20, 2023 (KR) 10-2023-0008847
May 24, 2023 (KR) 10-2023-0067544

(51) Int. Cl. C07K 16/46; C07K 14/47; C07K 14/52

(52) U.S. Cl. CPC C07K 16/46; C07K 14/47; C07K 14/522; C07K 2317/24; C07K 2317/52; C07K 2317/54

(57) ABSTRACT

The present invention relates to a conjugate in which a polypeptide capable of forming a dimer is fused to the C-terminus of the heavy chain or light chain of an antibody or fragment thereof, a composition for increasing antigen-binding avidity of an antibody in proportion to the density of a target antigen, and a method for increasing antigen-binding avidity using the same. The present invention also relates to the treatment or diagnosis of diseases using the conjugate.

### CROSS-REFERENCE TO RELATED APPLICATIONS

The present application is based on, and claims priority from, Korean Patent Applications No. 10-2022-0091716 filed on Jul. 25, 2022, No. 10-2023-0008847 filed on Jan. 20, 2023, and No. 10-2023-0067544 filed on May 25, 2023, the disclosures of which are hereby incorporated by reference herein in its entirety.

### SEQUENCE LISTING

The present application contains a Sequence Listing which has been submitted electronically in XML format and is incorporated herein by reference in its entirety. Said XML copy, created on Jul. 24, 2023, is named “97L9108.XML” and is 4,301 bytes in size. The Sequence Listing does not go beyond the disclosure of this application as filed.

### BACKGROUND OF THE INVENTION

Immunoglobulin G (IgG) antibodies are widely used biologics for diagnosis and treatment. IgG antibodies consist of two full length light chains and two full length heavy chains. In particular, IgG antibodies are a homodimer of a heterodimer composed of two copies of each heavy chain (˜50 kDa) and light chain (˜25 kDa). They have two functional regions: the antigen-binding fragment (Fab) region at the N-terminal end and the fragment crystallizable (Fc) region at the C-terminal end. With an overall shape of the letter Y, the two identical regions of Fab form two arms that bind to two antigen molecules. Fab includes a complementarity determining region (CDR) that binds to an antigen, and this antibody-antigen engagement could prevent the antigen from binding to cognate partners or eliminate the antigen molecules from the cell surface by receptor-mediated endocytosis. The two copies of Fc form a homodimeric tail that enables long half-life via binding to the neonatal Fc receptor (FcRn) and exerts effector functions via binding to the Fc receptors on effector immune cells or complement factor Clq of Cl. These interactions elicit the antibody-dependent cellular cytotoxicity (ADCC), antibody-dependent cellular phagocytosis (ADCP) or complement-dependent cytotoxicity (CDC), leading to the death of cells to which antibody molecules are bound. IgG antibodies have desirable properties for use as a diagnostic or therapeutic agent, including high specificity for a target antigen, low immunogenicity and long serum half-life. Thus, most diagnostic or therapeutic antibodies are in the form of IgG.

In general, diagnostic or therapeutic antibodies should have a high antigen-binding affinity (K_{D}<10 nM). However, therapeutic monoclonal antibodies (mAbs) often show a limited and only a moderate efficacy due to insufficient blockade of target antigens for various reasons including insufficient antigen-binding affinity. Further, although there is a difference in quantity, the target antigen is not expressed only on target cells but also on normal cells. Thus, antibodies may act on normal cells, which is a major cause of side effects.

Therefore, there is a demand for developing a technology for increasing the level of antigen-binding avidity of an antibody, particularly a technology capable of increasing the antigen-binding avidity of an antibody in proportion to the density of the antigen.

### SUMMARY OF THE INVENTION

The present invention relates to a technology to fuse proteins capable of forming homodimers to each of the two heavy chain C-termini or to each of the two light chain C-termini of an IgG type antibody or fragment thereof, which is capable of dramatically increasing the antigen-binding avidity of an antibody compared to the intrinsic binding affinity on the surface where target antigens are present.

An embodiment described herein provides a conjugate in which proteins capable of forming homodimers are fused to each of the two heavy chain C-termini or to each of the two light chain C-termini of an antibody or a fragment thereof.

Another embodiment described herein provides a composition for enhancing antigen-binding avidity of an antibody, the composition comprising the above conjugate.

Another embodiment described herein provides a method for enhancing antigen-binding avidity of an antibody, comprising treating the above conjugate on the surface where target antigens to which the antibody specifically binds are present.

Another embodiment described herein provides a biosensor for sandwich immunoassay comprising the above conjugate as a secondary antibody in sandwich immunoassay.

Another embodiment described herein provides a composition for diagnosing or treating a disease comprising the above conjugate.

Another embodiment described herein provides a method for diagnosing or treating a disease using the above conjugate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A to FIG. 1E. The concept of antibody catenation on a target surface by fusion of a catenator

FIG. 1A: Molecular model for catenator-fused antibodies. A flexible linker (Gly-Gly-Ser) between Fc and the catenator was modeled by using the ROSETTA software. The catenator is an α-helical hairpin that forms four-helix anti-parallel coiled coils (PDB entry: 1ROP). The structure of Fc was derived from the IgG1 antibody (PDB entry: 1IGY) and that of Fab from an antibody against the receptor-binding domain of the SARS-CoV-2 spike protein (PDB entry: 6XE1).

FIG. 1B: Decreased dissociation rate by antibody catenation when catenators are fused to each of the C-termini of the two heavy chains of the antibody. Pairs of ^{cat}Ab-antigen complexes adjacent to each other can be catenated, and the ^{cat}Ab molecules are increasingly harder to dissociate from each other with increased catenation. The effective antigen-binding avidity would increase owing to a decreased off rate of ^{cat}Ab.

FIG. 1C: Decreased dissociation rate by antibody catenation when catenators are fused to each of the C-termini of the two light chains of the antibody.

FIG. 1D: Decreased dissociation rate by antibody catenation when catenators are fused to each of the C-termini of the two heavy chains of the antibody in an antibody-drug conjugate (ADC).

FIG. 1E: Decreased dissociation rate by antibody catenation when catenators are fused to each of the C-termini of Fc fragment covalently linked to a therapeutic protein.

FIG. 2. Examples of catenators that form homodimers 3D modeling structure of SDF-1α(8-67), Sly1(254-316), HomoCC and (K_{D})_{catenator} value of each homodimer

FIG. 3A to FIG. 3C. Agent-based modeling (ABM) for simulating the binding dynamics of a catenator-fused antibody

FIG. 3A: (Left) Each binding site is composed of two antigen molecules (2Ag). (Right) The grey circles indicate the sphere sampled by the catenator, and Vₒᵥₑᵣₗₐₚ is the overlapping volume between the adjacent spheres. Catenation between two ^{cat}Ab molecules is possible only in Vₒᵥₑᵣₗₐₚ.

FIG. 3B: The three rules of the ABM model. (Left) ^{cat}Ab-2Ag binding occurs with a relative likelihood, which is determined by [^{cat}Ab] and K_{D}. (Middle) The catenation between adjacent ^{cat}Ab-2Ag complexes occurs with an indicated relative likelihood, which is determined by ƒ(d)/(K_{D})_{catenator}, which is affected by (K_{D})_{Catenator} and the inter-complex distance d. (Right) It was assumed that ^{cat}Ab molecules that are catenated cannot dissociate from the surface.

FIG. 3C: The simulation requires specification of the parameters for the binding site, antibody and catenator. Through the MCMC sampling, the state of binding sites on the target surface is iteratively updated with the ABM rules and eventually sampled. A sufficient number of sampling results are collected to quantify the binding occupancy and the effective dissociation constant.

FIG. 4A and FIG. 4B. Calculation of ƒ(d) using uniform local density approximation.

FIG. 4A: The forward catenation rate at which two catenators dimerize is proportional to the volumetric overlap (V(d)) between the effective concentration of the catenator, which is assumed to be uniformly distributed over a sphere defined by the reach length (L). V(d) depends on the distance (d) between the two adjacent ^{cat}Ab-2Ag complexes as well as the reach length.

FIG. 4B: It indicates a plot of ƒ(d) as a function of d calculated for the five indicated reach length (L).

FIG. 5. Simulations of the binding site occupancy and (K_{D})_{eff} in response to (K_{D})_{catenator}.

(Left) Binding site occupancy. The simulations were carried out with a square array of the binding sites. The values for a set of variables were K_{D}=10⁻⁸ M, [^{cat}Ab]=10⁻⁹ M, reach length=7 nm, spacing between the binding sites=12 nm and the number of total binding sites=98. The mean value and standard deviations of 1024 MCMC simulations for each (K_{D})_{catenator} value are shown in blue, and the data are shown as a scatter plot of representative runs (orange).

(Right) The effective dissociation constant. The data shown on the left were converted into the (K_{D})_{eff} values. The dashed line represents the K_{D} value for the same antibody without the catenator. The maximum fold enhancement of the effective binding avidity, which is equivalent to the reduction of (K_{D})_{eff}, is 70.6.

FIG. 6A and FIG. 6B. Simulations of different arrays of the binding sites.

FIG. 6A: Comparison for regularly distributed binding sites. Three different regular arrays of the binding sites are shown at the top. The black dots represent the binding sites and grey lines the connectable pairs by the catenators. The red circles and the blue lines represent the maximum range of catenation and the connectivity number, respectively, for a given binding site. Binding site occupancy and (K_{D})_{eff} in response to (K_{D})_{catenator} are shown at the bottom. 1024 trials were sampled for each (K_{D})_{catenator} value and the results are plotted. The variables were K_{D}=10⁻⁸ M, [^{cat}Ab]=10⁻⁹ M, reach length=7 nm, spacing between the binding sites=12 nm, and the number of total binding sites were 98 for the square array and 102 for hexagonal and triangular array, respectively. The numbers on the right are the maximum fold enhancement of the effective binding avidity for each array.

FIG. 6B: Comparison for randomly distributed binding sites. Three random arrays of the binding sites with different binding site density (p) are shown at the top. The surface area for the simulation was 5,760 nm². The simulation conditions were the same as in (A). Binding site occupancy and (K_{D})_{eff} in response to (K_{D})_{catenator} are plotted as in (A).

FIG. 7. Simulations for randomly distributed, high-density binding sites.

1024 trials were sampled for each (K_{D})_{catenator} value at the indicated density and the results are plotted. The variables were K_{D}=10−8 M, [^{cat}Ab]=10⁻⁹ M, reach length=7 nm, spacing between the binding sites=12 nm, and the surface area=5,760 nm². The maximum fold enhancement of the effective binding avidity and (K_{D})_{catenator} are tabulated at the bottom.

FIG. 8A and FIG. 8B. Influence of the likelihood of intrinsic antigen binding ([^{cat}Ab]/K_{D}) on binding site occupancy and (K_{D})_{eff}.

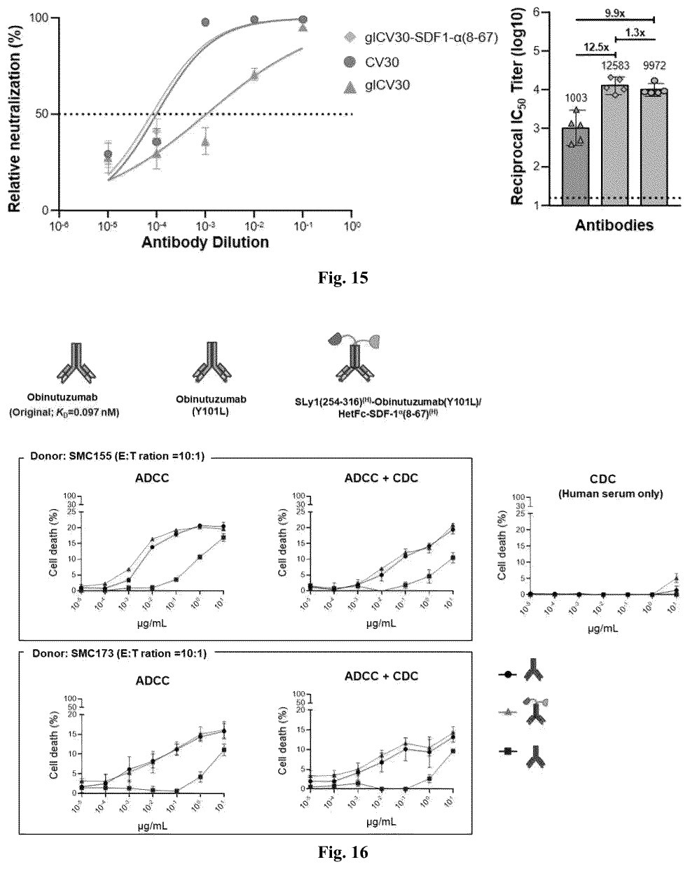

The binding occupancy (FIG. 8A) and the effective dissociation constant (K_{D})_{eff} (FIG. 8B) in response to [^{cat}Ab]/K_{D} for [^{cat}Ab]/K_{D}=1.0, 0.3, 0.1, 0.03, and 0.01. In FIG. 8A and FIG. 8B, the simulations were carried out with a square array of the binding sites as in FIG. 5. The set values for the variable parameters were [^{cat}Ab]=10⁻⁹ M, reach length=7 nm, spacing between the binding sites=12 nm and the number of total binding sites=98. The mean binding occupancy of 1024 MCMC simulations was plotted. (K_{D})_{catenator} was varied from 3 mM to 30 nM. The antibody binding avidity is substantially enhanced across a broad range of [^{cat}Ab]/K_{D}.

FIG. 9A and FIG. 9B. BLI runs demonstrating the effect of catenation on the binding avidity (I).

The binding kinetics were measured by BLI with the indicated targets immobilized on a sensor tip. The concentration of the antibodies was varied as shown. The experimental signals and fitted curves are shown in red and black, respectively. For curve fitting, 1:1 binding was assumed. The kinetic parameters are shown in the insets. kₐ, association rate constant; k_{d}, dissociation rate constant.

FIG. 9A: High affinity anti-HER2 antibody. Trastuzumab(N30A/H91A) exhibited the K_{D} of 2.1 nM for the immobilized ectodomain of HER2.

FIG. 9B: Low affinity anti-SARS-CoV-2 antibody. glCV30 exhibited the K_{D} of 1.3 nM for RBD of SARS-CoV-2. For all antibodies fused with SDF-1α, the K_{D} values could not be accurately determined due to the instrumental insensitivity (K_{D}<0.01 nM). The experiments were performed in triplicates, and representative sensorgrams are shown.

FIG. 10A and FIG. 10B. BLI runs demonstrating the effect of catenation on the binding avidity (II).

The binding kinetics were measured by BLI. The concentration of the antibodies was varied as shown. The experimental signals and fitted curves are shown in red and black, respectively. For curve fitting, 1:1 binding was assumed.

Trastuzumab(N30A/H91A)-SDF-1α(8-67)^{(L)} (FIG. 10A) and Trastuzumab(N30A/H91A)-HomoCC^{(H)} (FIG. 10B) were reacted with the HER ectodomain immobilized on a sensor tip. For both antibody-catenators, the K_{D} values could not be accurately determined due to the instrumental insensitivity (K_{D}<0.01 nM). The experiments were performed in triplicates, and representative sensorgrams are shown.

FIG. 11A to FIG. 11C. BLI runs demonstrating the effect of catenation on the binding avidity (III).

Trastuzumab (N30A/H91A/Y100A) was prepared by introducing three mutations into Trastuzumab so that the increased actual K_{D} value did not exceed the instrumental measurement limit (0.01 nM), and then, SLy1(254-316)^{(H)}-Trastuzumab(N30A/H91A/Y100A)/HetFc-SDF-1α(8-67)^{(H)}, a construct in which two catenators were fused, was prepared using heterodimeric Fc (HetFc). In addition, SLy1(254-316)^{(H)}-Trastuzumab(N30A/H91A/Y100A)/HetFc, a construct in which only one catenator was fused, was prepared for a control experiment. In each construct, mScarlet was fused to the C-terminus of the light chain.

FIG. 11A: Elution of Trastuzumab(N30A/H91A/Y100A), SLy1(254-316)^{(H)}-Trastuzumab(N30A/H91A/Y100A)/HetFc-SDF-1α(8-67)^{(H)}, and SLy1(254-316)^{(H)}-Trastuzumab(N30A/H91A/Y100A)/HetFc in monomeric size from the size-exclusion column. An inverted triangle represents the peak position of the size-marker. The right side is the image of SDS-PAGE (Sodium dodecyl sulfate polyacrylamide gel electrophoresis), showing the purity of the indicated proteins. Trastuzumab(N30A/H91A/Y100A) was denoted Trz(N30A/H91A/Y100A), and mScarlet fused to the C-terminus of the light chain was denoted as L-mScarlet.

FIG. 11B: Catenation between antibodies having heterodimeric Fc and two different catenators on the target surface. Antibodies having one catenator cannot be catenated (Right).

FIG. 11C: Trastuzumab(N30A/H91A/Y100A), SLy1(254-316)^{(H)}-Trastuzumab(N30A/H91A/Y100A)/HetFc-SDF-1α(8-67)^{(H)}, or SLy1(254-316)^{(H)}-Trastuzumab(N30A/H91A/Y100A)/HetFc were reacted with the HER2 ectodomain immobilized on a sensor tip. The fold increase compared to the antigen-binding avidity of the mother antibody is indicated in red letters. When only one catenator was fused, the binding avidity was not increased.

FIG. 12A and FIG. 12B. The effect of catenation for Obinutuzumab(Y101L) SLy1(254-316)^{(H)}-Obinutuzumab(Y101L)/HetFc-SDF-1α(8-67)^{(H)}, in which two catenators were fused to the parental antibody Obinutuzumab (Y101L), was prepared using HetFc.

FIG. 12A: The binding kinetics were measured by BLI. The concentration of the antibodies was varied as shown. The experimental signals and fitted curves are shown in red and black, respectively. For curve fitting, 1:1 binding was assumed. Obinutuzumab(Y101L) or SLy1(254-316)^{(H)}-Obinutuzumab(Y101L)/HetFc-SDF-1α(8-67)^{(H)} were reacted with CD20 immobilized on a sensor tip. The fold increase compared to the antigen-binding avidity of the mother antibody is indicated in red letters. SLy1(254-316)^{(H)}-Obinutuzumab(Y101L)/HetFc-SDF-1α(8-67)^{(H)} bound to CD20 much better than the mother antibody Obinutuzumab (Y101L).

FIG. 12B: Flow cytometry analysis of the degree of binding of the two antibodies to SU-DHL5, a type of B cell. Both antibodies were labeled with muGFP at the C-terminus of the light chain. The fluorescence signal of muGFP was detected using a 525/40 bandpass filter.

FIG. 13. Antibody catenation increases binding avidity to breast cancer cells in proportion to the level of HER2 expression.

mScarlet fluorescent protein was tagged to the light chain C-terminus of the antibodies for fluorescence microscopy.

(Top Left) Western blotting analysis showing that the four indicated breast cell lines express HER2 at different levels.

(Bottom Left) Cells were seeded in two separate wells, and Ab (Trastuzumab(N30A/H91A)) or ^{cat}Ab (Trastuzumab (N30A/H91A)-SDF-1α(8-67)^{(H)}) was added to the wells right before confocal imaging.

(Right) Confocal images were obtained 30 min after the addition of antibodies.

FIG. 14. Trastuzumab(N30A/H91A)-SDF-1α(8-67)^{(H)} binds preferentially to BT-474 cells with high HER2 expression levels rather than to MCF-7 cells with low HER2 expression levels.

GFP fluorescent protein was fused to the light chain C-terminus of the antibodies for fluorescence microscopy.

(Top Left) Western blotting analysis showing that the four indicated breast cell lines express HER2 at different levels. MCF-7 cells express HER2 at lower level than BT-474 cells.

(Bottom Left) The two indicated cell lines were seeded separately, and the media containing Ab (Trastuzumab(N30A/H91A)) or ^{cat}Ab (Trastuzumab (N30A/H91A)-SDF-1α(8-67)^{(H)}) was added right before confocal imaging.

(Right) Confocal images were obtained 30 min after the addition of antibodies.

FIG. 15. Fusion of the catenator to gICV30 greatly enhances virus neutralization activity.

(Left) Neutralization of vesicular stomatitis virus (VSV) pseudotyped with the SARS-CoV-2 spike protein. Each of the three antibodies (CV30, glCV30 and glCV30-SDF-1α(8-67)) was serially diluted and added to rVSV-ΔG-Luc containing the SARS-CoV-2 spike protein of Wuhan-Hu-1 strain. The mixture was incubated with HEK293T-hACE2 cells and luciferase activity was measured.

(Right) Geometric mean titer (GMT) values with 95% confidence intervals are shown on the right, corresponding to IC₅₀ values of 0.25, 3.22 and 0.21 μg/ml for CV30, glCV30 and glCV30-SDF-1α, respectively.

FIG. 16. Effector function of Obinutuzumab(Y101L)-catenator

SLy1(254-316)^{(H)}-Obinutuzumab(Y101L)/HetFc-SDF-1α(8-67)^{(H)}, in which two catenators were fused to the mother antibody Obinutuzumab (Y101L), was prepared using HetFc. The original Obinutuzumab was also prepared. Antibody-dependent cytotoxicity (ADCC) and complement-dependent cytotoxicity (CDC) of Obinutuzumab, Obinutuzumab(Y101L), or SLy1(254-316)^{(H)}-Obinutuzumab(Y101L)/HetFc-SDF-1α(8-67)^{(H)} were measured using the Incucyte® Live-Cell Analysis System. To measure ADCC, peripheral blood mononuclear cells (PBMC) extracted from the blood of two donors (marked as Donor) were used respectively (Effector cell:Target cell ratio=10:1).

### DETAILED DESCRIPTION OF THE INVENTION

Overview

Given the unique dimeric structure of IgG, the present inventors hypothesized that, by genetically fusing a homodimer-forming protein (‘catenator’) to each of the two C-termini of the heavy chain or to each of the two C-termini of the light chain, reversible linkage (‘catenation’) of antibody molecules could be induced on a surface where target antigen molecules are abundant, and that it could be an effective way to greatly enhance the antigen-binding avidity. This enhancement of the binding affinity arises from the ‘proximity effect’, where the binding of one subunit of the dimer to a target restricts the search space for the other subunit.

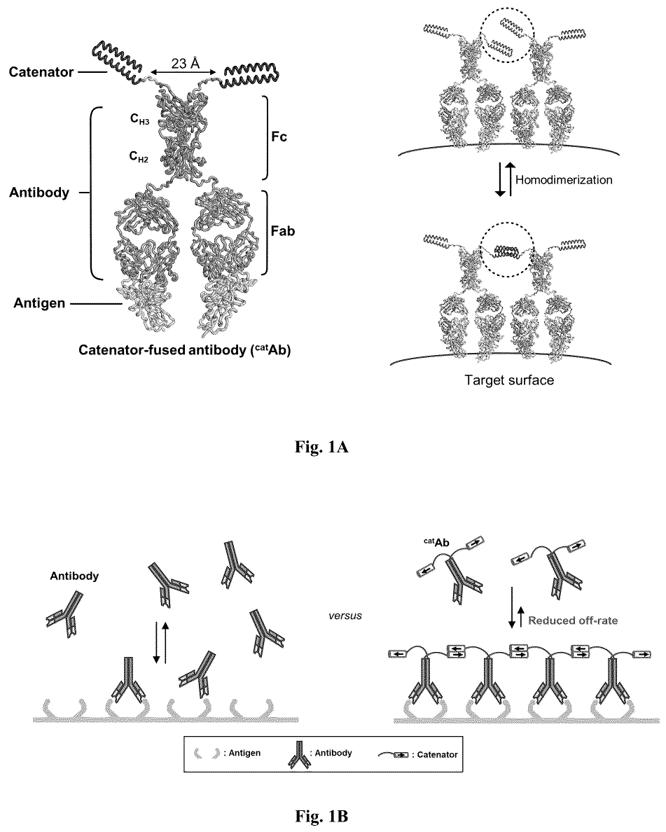

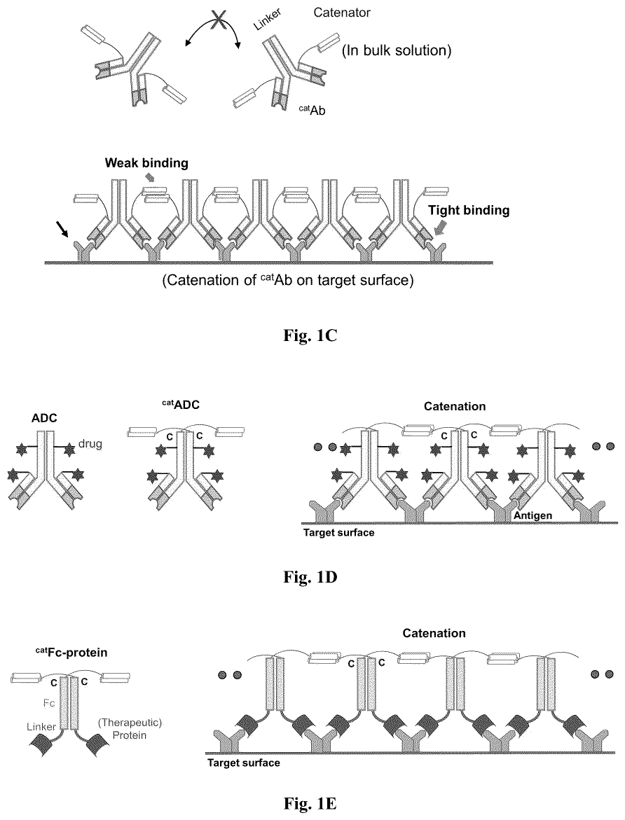

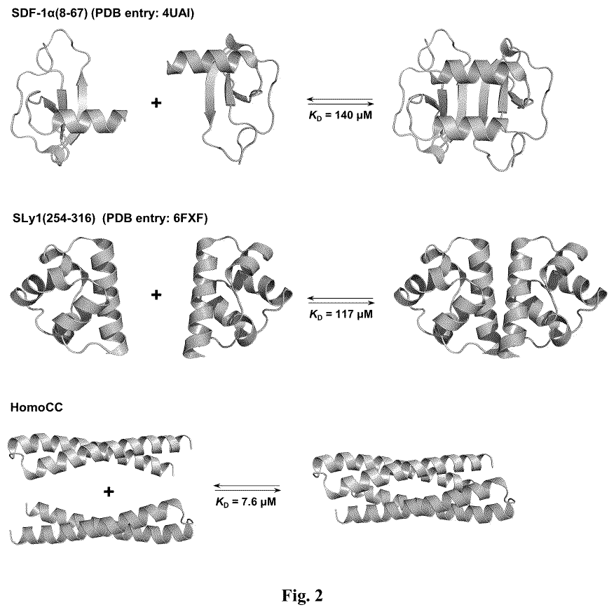

Specifically, owing to the overall dimeric structure, IgG antibodies in which homodimer-forming proteins (catenators) is fused to each of the two C-termini of the heavy chain or to each of the two C-termini of the light chain can be catenated in an arm-in-arm fashion as long as the homodimer can be formed, not within an antibody molecule, but between two antibody molecules. In this regard, the present inventors found that if catenators having a quite low homodimerization affinity are fused to each of the two heavy chain C-termini or to each of the two light chain C-termini, the catenators remain in a monomeric state in solution, but on a cell surface where target antigen molecules are abundant, the catenators form homodimers due to the proximity effect, thereby forming carnations between the antibody-catenator fusion proteins. Further, in the structure of IgG, the crystallizable fragment (Fc) is composed of two copies of the constant regions of the heavy chain (C_{H2} and C_{H3}) forming a homodimer, in which the two C-termini are ˜23 Å apart and point away from each other (FIG. 1A, Left). This structural feature indicated that a catenator, which has a very low homodimerization affinity and is genetically fused to the C-terminus of an antibody, can be prevented from forming a homodimer intramolecularly. Instead, as there is no physical barrier in intermolecular space between antibodies, the fusion proteins in adjacent can form a homodimer intermolecularly, and then such a homodimerization could result in a catenation of the antibody molecules (FIG. 1A, Right). In particular, on the target surface where multiple copies of target antigen are present, the local concentration of the catenator-fused antibody increases due to the binding between the antigen and the antibody, and the concentration of the catenator also increases. Therefore, the formation of homodimers between catenators increases due to the proximity effect, and as a result, antibody molecules can be catenated in an arm-in-arm fashion on the target surface (FIG. 1B and FIG. 1C). That is, in a solution other than the target surface, the catenator-fused antibodies are not catenated, but can be catenated on the target surface where multiple copies of target antigen are present. When such catenation is established, the dissociation rate of the catenator-fused antibody from the antigen is decreased and the effective binding affinity of the antigen-binding site can be increased.

A thermodynamic simulation provided herein shows that quite low homodimerization affinity of a catenator (e.g. dissociation constant of 0.1 μM to 500 μM) can enhance nanomolar antigen-binding avidity to a picomolar level, and that the fold enhancement sharply depends on the concentration of the antigen (FIG. 2 to FIG. 8). In a proof-of-concept experiment where a biosensor tip is immobilized with antigen molecules, C-terminal fusion of catenators having a weak homodimerization affinity to four different antibodies enhanced the antigen-binding avidity by at least 100 to 304 folds from the intrinsic binding avidity (FIG. 9 to FIG. 12). Furthermore, in the analysis of the characteristics of catenated antibodies using cancer cell lines, the binding avidity of antibody-catenator to the antigen increased in proportion to the density of the antigen, and it selectively bound to cancer cells expressing more of the antigen (FIG. 13 to FIG. 16).

Thus, the present invention was completed by confirming that antibody catenation induced by such intermolecular homodimerization can be a simple but powerful and general approach for many antibody applications, including detection of scarce biomarkers and anticancer therapies.

According to one aspect of the present invention, there is provided a conjugate comprising (i) an antibody or fragment thereof, and (ii)_{catenator} polypeptides fused to each of the two heavy chain C-termini or to each of the two light chain C-termini of the antibody or fragment thereof. Preferably, the catenator polypeptide refers to a polypeptide that satisfies one or more of the following criteria:
(a) is capable of forming a dimer;
(b) the dissociation constant (K_{D}) of dimer formation is between 0.1 μM and 500 μM;
(c) a molecular weight is between 3 kDa and 30 kDa; and
(d) On the surface where target antigens to which the antibody specifically binds are present, pairs of the conjugate-antigen complexes adjacent to each other are catenated by intermolecular dimerization between catenator polypeptides.

According to another aspect of the present invention, there is provided a composition for enhancing antigen-binding avidity of an antibody, the composition comprising the above conjugate, which is characterized in that, on the surface where target antigens to which the antibody included in the conjugate specifically binds are present, pairs of the conjugate-antigen complexes adjacent to each other are catenated by intermolecular dimerization between catenator polypeptides, thereby suppressing dissociation of the antibody.

According to another aspect of the present invention, there is provided a method for enhancing antigen-binding avidity of an antibody, comprising treating the above conjugate on the surface where target antigens to which the antibody specifically binds are present, which is characterized in that, on the surface, pairs of the conjugate-antigen complexes adjacent to each other are catenated by intermolecular dimerization between catenator polypeptides, thereby suppressing dissociation of the antibody.

According to another aspect of the present invention, there is provided a biosensor for sandwich immunoassay comprising the above conjugate as a secondary antibody in sandwich immunoassay.

According to another aspect of the present invention, there is provided a composition for diagnosing or treating a disease comprising the above conjugate.

According to another aspect of the present invention, there is provided a method for diagnosing or treating a disease using the above conjugate.

Hereinafter, the present invention will be described in more detail.

The term “conjugate” is used in its general meaning in the art and refers to a covalent or non-covalent complex, preferably to a covalent complex and most preferably to a fusion protein. For example, “conjugate” refers to a polypeptide formed by the joining of two or more polypeptides through a peptide bond formed between the amino terminus of one polypeptide and the carboxyl terminus of another polypeptide. The conjugate may be formed by the chemical coupling of the constituent polypeptides or it may be expressed as a single polypeptide fusion protein from a nucleic acid sequence encoding the single contiguous conjugate.

As used herein, the terms “comprise”, “comprises”, “comprised” or “comprising” are to be interpreted as specifying the presence of the stated features, integers, steps or components, but not precluding the presence of one or more other features, integers, steps or components or groups thereof.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention before the priority date of each claim of this application.

Homodimer-forming protein (Catenator)

The term “homodimer” as used herein refers to a complex formed by the interaction between two identical monomeric proteins. On the other hand, the term “heterodimer” refers to a complex formed by the interaction of two different monomeric proteins.

The term “homodimer-forming protein” or “a protein capable of forming a homodimer” refers to a monomeric protein capable of forming homodimer. It is used herein to catenate antibody molecules by homodimer formation, and is therefore also called “catenator” or “catenator polypeptide”.

The catenator satisfies one or more of the following criteria:
(a) is capable of forming a dimer;
(b) the dissociation constant (K_{D}) of dimer formation is between 0.1 μM and 500 μM;
(c) a molecular weight is between 3 kDa and 30 kDa; and
(d) On the surface where target antigens to which the antibody specifically binds are present, pairs of the conjugate-antigen complexes adjacent to each other are catenated by intermolecular dimerization between catenator polypeptides.

The criterion (a) is based on the technical idea of the present invention to form catenated antibodies in an arm-in-arm fashion by the homodimerization between the catenator molecules fused to each antibody, thereby increasing the antigen-binding avidity of an antibody to the target antigen. Thus, the catenator of the present disclosure should be capable of forming a homodimer when present in close proximity to each other under physiological conditions.

The criterion (b) means that the catenator should have low homodimerization affinity. The catenator herein is fused to the C-terminus of either the heavy or light chain of the antibody. Typically, IgG antibodies consist of two heavy chains and two light chains, so the antibody has two heavy chain C-termini and two light chain C-termini to the antibody. Accordingly, since the catenator described herein may be fused to each of the two heavy chain C-termini or to each of the two light chain C-termini, one antibody may have at least two catenators. However, with respect to the objects of the present invention to catenate antibody molecules by homodimerization between catenators, it is necessary to prevent the formation of homodimers between catenators within one antibody molecule (i.e., intramolecular homodimerization) and instead to form homodimers between catenators between antibody molecules (i.e., intermolecular homodimerization). Given that the two C-terminal domains of the heavy chain or the two C-terminal domains of the light chain are physically constrained to be far apart from each other, if the catenator has sufficiently low homodimerization affinity, the formation of homodimers between catenators within one antibody molecule can be prevented due to the physical constraint. Thus, while in solution (e.g., blood) the catenators remain monomeric (i.e., antibody-catenator fusions remain), on the surface where target antigen molecules are abundant, the catenators form homodimers due to the proximity effect, thereby forming carnations between the antibody-catenator fusion proteins.

In this regard, given the physical distance of the two heavy chain C-terminal domains or the two light chain C-terminal domains of the antibody, if the dissociation constant (K_{D}) of homodimer formation of the catenator is 0.1 μM or more and 500 μM or less, the catenator can be considered to have a sufficiently low homodimerization affinity to prevent intramolecular catenation. For example, the dissociation constant (K_{D}) of homodimer formation of the catenator may be 0.1 μM or more, 1 μM or more, 10 μM or more, 20 μM or more, 30 μM or more, 40 μM or more, 50 μM or more, 60 μM or more, 70 μM or more, 80 μM or more, 90 μM or more, or 100 μM or more; and 500 μM or less, 400 μM or less, 300 μM or less, or 200 μM or less, but is not limited thereto.

The term “affinity” refers to the equilibrium constant for the reversible binding of two agents and is expressed as the dissociation constant (K_{D}). The affinity can be measured using any method known in the art. Such methods include, for example, fluorescence activated cell sorting (FACS), surface plasmon resonance (e.g., Biacore, ProteOn), biolayer interferometry (BLI, e.g. Octet), kinetics exclusion assay (e.g. KinExA), separable beads (e.g., magnetic beads), antigen panning, and/or ELISA. It is known in the art that the binding affinity of a particular antibody will vary depending on the method that is used to analyze the binding affinity.

The criterion (c) means that it is preferable that the size of the catenator is small so as not to cause immunogenicity and not to substantially affect the physical properties of the antibody. In this respect, if the molecular weight of the catenator is 3 kDa or more and 30 kDa or less, the catenator can be considered to have a sufficiently small size so as not to cause immunogenicity or affect the physical properties of the antibody while exhibiting a function as a catenator. For example, the molecular weight of the catenator may be 3 kDa or more, 4 kDa or more, 5 kDa or more, 6 kDa or more or 7 kDa or more; and 30 kDa or less, 25 kDa or less, 20 kDa or less, 19 kDa or less, 18 kDa or less, 17 kDa or less, 16 kDa or less, 15 kDa or less, 14 kDa or less, 13 kDa or less, 12 kDa or less, 11 kDa or less, or 10 kDa or less, but is not limited thereto.

The criterion (d) relates to the fact that on the target surface where target antigens to which the antibody specifically binds are present, the local concentration of the catenator-fused antibody increases due to the binding between the antigen and the antibody, and the concentration of the catenator also increases, and thus, the formation of homodimers between catenators increases due to the proximity effect, and as a result, antibody molecules can be catenated long like a chain in an arm-in-arm fashion on the target surface, thereby suppressing dissociation of the antibody and greatly increasing the antigen-binding avidity of an antibody.

The term “surface” means any surface, whether interior or exterior, vertical or horizontal, in vivo or in vitro, of any body or object. For example, the surface may be a surface of a biological material such as cells or a surface of a scaffold. For example, the surface may be a surface of a cell expressing a target antigen (e.g., a surface of a cancer cell expressing a tumor antigen) or a surface of a cell expressing a target biomarker of disease diagnosis (e.g., a surface of a cell expressing a target biomarker protein). Alternatively, the surface may be, but is not limited to, a surface of a reaction vessel or support, such as a support of a metal, plastic, glass or ceramic component or of a polymeric or elastic support.

According to the examples described herein, as catenators that satisfy the above criteria, a polypeptide consisting of amino acid residues 8-67 of the human chemokine protein, stromal cell-derived factor-1a (SDF-1α) (SEQ ID NO: 1; Molecular weight 8 kDa, homodimerization K_{D} 140 μM); Sterile Alpha Motif domain (SAM) domain consisting of amino acid residues 254-316 of SH3 domain-containing protein expressed in lymphocytes 1 (SLy1) (SEQ ID NO: 2; Molecular weight 7.4 kDa, homodimerization K_{D} 117 μM); and a polypeptide named ‘homodimeric coiled-coil (HomoCC)’, a coiled-coil type protein designed de novo by the present inventors (SEQ ID NO: 3; Molecular weight 8.7 kDa, homodimerization K_{D} 7.6 μM). However, since these are merely provided as representative examples, the scope of the present invention should not be construed as being limited thereto.

TABLE 1

| | Amino acid sequence |
|---|---|
| Catenator | (Sequence ID Number) |
| SDF-1α | RCPCRFFESHVARANVKHLKILNTPACALQIVARLKNN |
| (8-67) | NEQVCIDPKLKWIQEYLEKALN (SEQ ID NO: 1) |
| SLy1 | KTLHELLERIGLEEHTSTLLLNGYQTLEDFKELRETHL |
| (254-316) | NELNIMDPQHRAKLLTAAELLLDYD (SEQ ID NO: |
| | 2) |
| HomoCC | DEEQRKVVEEDLKVLEHLRRVVERKEHLVRDAYEETFD |
| | DQQREVVREKLKVLEHLEKVIERDRHLSSRPGL (SEQ |
| | ID NO: 3) |

Antibody or Fragment Thereof

The term “antibody” is used herein in its broadest sense to refer to a protein that specifically binds to a specific antigen or epitope, and may be a protein produced by stimulation of an antigen in the immune system, or a protein produced by chemical synthesis or recombinant production, with no specific limitation. Specifically, the antibody encompasses monoclonal antibodies (including full-length monoclonal antibodies), polyclonal antibodies, multispecific antibodies (e.g., bispecific antibodies), synthetic antibodies (also called antibody mimetics), chimeric antibodies, humanized antibodies, human antibodies, or antibody fusion proteins (also called antibody conjugates), provided such antibodies exhibit a desired biological activity. The antibody may be a therapeutic antibody or a diagnostic antibody, but is not limited thereto.

The term “monoclonal antibody” refers to an antibody molecule obtained from a population of substantially homogeneous antibodies. The monoclonal antibody displays a single-binding specificity and affinity for a specific epitope. The term “polyclonal antibody” refers to an antibody mixture comprising two or more monoclonal antibodies that bind to different epitopes of the same antigen. The polyclonal antibody is capable of reacting with a plurality of epitopes. The term “chimeric antibody” refers to an antibody obtained by recombining the variable region of a non-human antibody and the constant region of a human antibody. The chimeric antibody provides greatly improved immune response compared to non-human antibody. The term “humanized antibody” refers to an antibody formed by grafting all or some of a CDR sequence of a non-human antibody into a human antibody. For example, CDRs of a murine monoclonal antibody may be recombined with human antibody-derived framework region (FR) to prepare a humanized variable region, and then the humanized variable region may be recombined with a constant region of a desired human antibody to prepare a humanized antibody, but the present invention is not limited thereto. The term “human antibody” refers to an antibody that is completely free of parts derived from non-human animals. In general, a human antibody refers to an antibody in which both constant region and variable region including CDR and FR regions are derived from human germline immunoglobulin sequences.

An intact antibody (e.g., IgG type) has a structure with two full-length light chains and two full-length heavy chains in a Y shape, and is a homodimer of heterodimers where each heterodimer is composed of one copy of the heavy chain and one copy of the light chain. Each light chain is linked to a corresponding heavy chain via a disulfide bond. The constant region of an antibody is divided into a heavy-chain constant region and a light-chain constant region. The heavy-chain constant region is of a gamma (γ), mu (μ), alpha (α), delta (δ), or epsilon (ε) type, and has gamma1 (γ1), gamma2 (γ2), gamma3 (γ3), gamma4 (γ4), alpha1 (α1) or alpha2 (α2) as its subclass. The light chain constant region is of either a kappa (κ) or lambda (λ) type.

As used herein, the term “heavy chain” may be intended to encompass a full-length heavy chains and fragments thereof, wherein the full-length heavy chain may comprise a variable region V_{H} including amino acid sequences sufficient to provide specificity to antigens, three constant regions C_{H1}, C_{H2}, and C_{H3}, and a hinge.

The term “light chain” may be intended to encompass full-length light chains and fragments thereof, wherein the full-length light chain may comprise a variable region V_{L} including amino acid sequences sufficient to provide specificity to antigens, and a constant region C_{L}.

The term “complementarity-determining region (CDR)” refers to a region in a variable region of an antibody, which imparts binding specificity or binding affinity to an antigen. Generally, there are three CDRs (CDR-H1, CDR-H2, CDR-H3) in a heavy chain variable region, and three CDRs (CDR-L1, CDR-L2, CDR-L3) in a light chain variable region. The CDRs may provide key contact residues for an antibody or a fragment thereof binding to an antigen or an epitope. The term “framework region (FR)” refers to non-CDR regions of variable regions of heavy and light chains. Generally, there are four FRs (FR-H1, FR-H2, FR-H3, and FR-H4) in a heavy chain variable region, and four FRs (FR-L1, FR-L2, FR-L3 and FR-L4) in a light chain variable region. The precise amino acid sequence boundaries of a given CDR or FR may be readily determined using any of a number of well-known schemes, such as Kabat numbering scheme, Chothia numbering scheme, Contact numbering scheme, IMGT numbering scheme, Aho numbering scheme, AbM numbering scheme, etc.

The term “variable region” refers to a domain of a heavy chain or light chain of an antibody, which is involved in binding of the antibody to an antigen. The heavy chain variable (V_{H}) region and the light chain variable (V_{L}) region generally have the similar structure, and each domain includes four conserved framework regions (FRs) and three CDRs.

The term “fragment of an antibody” refers to a any fragment of an antibody that lacks at least some of the amino acids present in the full-length chain of the antibody. With respect to the objects of the present invention, the antibody fragment may be a fragment that maintains the unique homodimer form of the antibody, and may be, for example, Fc or F(ab′) 2, but is not limited thereto.

F(ab′) 2 generally refers to a fragment containing an antigen-binding site among fragments generated by digestion of an antibody with a proteolytic enzyme, pepsin. It is a homodimeric form in which two heterodimeric Fabs composed of light chain V_{L} and V_{H} domains and heavy chain C_{L} and C_{H1} domains are linked by a disulfide bond in the hinge region. Fc region is in the form of a dimer of a heavy chain fragment composed of heavy chain C_{H2} and C_{H3} domains, and may include a hinge portion in some cases.

By genetically fusing a homodimer-forming protein (‘catenator’) to each of the two C-termini of the heavy chain or to each of the two C-termini of the light chain of the antibody or a fragment thereof, reversible linkage (‘catenation’) of antibody molecules can be induced on a surface where target antigens are present, thereby dramatically increasing the antigen-binding avidity of an antibody compared to the intrinsic binding affinity.

Therefore, this technical idea is applicable to various antibody fields using antibodies or fragments thereof in which all or part of the unique homodimeric structure of the antibody is maintained, and some of them are exemplified as follows.

Antibody-Drug Conjugate (ADC)

The term “antibody-drug conjugate, ADC” refers to an antibody to which a therapeutically active substance or an active pharmaceutical ingredient (API) has been covalently coupled, such that the therapeutically active substance or an active pharmaceutical ingredient (API) can be targeted to the binding target of the antibody to exhibit its pharmacologic function. The therapeutically active substance or an active pharmaceutical ingredient may be a cytotoxin that is able to kill malignant or cancer cells, or an antibiotic, an enzyme such as nuclease, or a radionuclide, but is not limited thereto. The covalent attachment of a therapeutically active substance or an active pharmaceutical ingredient may be performed in a non-site specific manner using standard chemical linkers that couple them to lysine or cysteine residues, or, preferably the conjugation is performed in a site-specific manner, that allows full control of conjugation site and drug-to-antibody ratio (DAR) of the ADC to be generated.

The terms “cytotoxin” and “cytotoxic agent” refer to any molecule that inhibits or prevents the function of cells and/or causes destruction of cells (cell death), and/or exerts antiproliferative effects. It will be appreciated that a cytotoxin or cytotoxic agent of an ADC also is referred to in the art as the “payload” of the ADC. A number of classes of cytotoxic agents are known in the art to have potential utility in ADC molecules and can be used in the ADC described herein. Such classes of cytotoxic agents include, microtubule structure formation inhibitors, meiosis inhibitors, topoisomerase inhibitors, or DNA intercalators, but are not limited thereto. Examples of specific cytotoxic agents include maytansinoid, auristatin, dolastatin, trichothecene, CC-1065 drug (NSC 298223), calicheamicin, enediynes, taxane, anthracycline, methotrexate, adriamycin, vindesine, vinca alkaloid, doxorubicin, melphalan, mitomycin C, chlorambucil, daunorubicin, daunomycin, etoposide, teniposide, carminomycin, aminopterin, dactinomycin, bleomycin, esperamicin, 5-fluorouracil, melphalan, nitrogen mustar (mechlorethamine HCL), cis-platinum and its analogs, cisplatin, CPT-11, doxorubicin, or docetaxel, but are not limited thereto.

As for the “antibody” of the antibody-drug conjugate herein, the features for the antibody as described above may be applied. The above described catenators may be fused to each of the two heavy chain C-termini or to each of the two light chain C-termini of the antibody or fragment thereof, and thus, on a surface where target antigens are present, homodimers are formed between catenators to link antibody-drug conjugates to each other, thereby enhancing antigen-binding avidity of an antibody and improving the pharmacological function of the antibody-drug conjugate (FIG. 1D).

Multispecific Antibody

The term “multispecific antibody” refers to an antibody having two or more antigen-binding sites, at least two of which bind to different antigens or to different epitopes of the same antigen. For example, “bispecific antibody” refers to an antibody having two different antigen-binding specificities, and “trispecific antibody” refers to an antibody having three different antigen-binding specificities.

Multispecific antibodies may have various formats depending on how two or more antibodies are combined. With respect to the objects of the present invention, the multispecific antibody is preferably of the IgG-like type structure, having a homodimeric or heterodimeric Fc region as a common basis.

In particular, the Fc region of a multispecific antibody may be homodimeric or heterodimeric. In general, homodimerization of the Fc region is induced by a non-covalent bond between the last domains of an antibody constant region (C_{H3} domain in for IgG) and a disulfide bond between hinge regions. Heterodimeric Fc region can be generated through engineering so as to have a bond where heterodimerization is preferred and homodimerization is not preferred or inhibited, via a specific non-covalent bond between the last domains of a constant region that greatly contribute to homodimerization of naturally occurring antibodies. For example, through gene manipulation, mutations are induced in CH3 domains of two different antibody heavy chains so that the two heavy chains are very similar in structure to naturally occurring antibodies, have a minimal deviation in sequence, and form a heterodimer. Examples of technologies related to this include a knob-into-hole technology available from Genentech, ZW1 from Zymeworks, HA-TF available from XENECORE Co., Ltd, SEEDbody available from EMD Serono, and the like, but are not limited thereto.

An Fc-based multispecific antibody may have a bilaterally symmetrical structure or an asymmetrical structure. Symmetric Fc-based multispecific antibodies are usually larger than typical IgGs because variable region (Fv) or scFv portion having different antigenic specificity is added as an antigen binding site to the N-terminus or C-terminus of the light chain or heavy chain of IgG. Instead of Fv or scFv, domain antibodies or alternative binding scaffold molecules may be used. Asymmetric Fc-based multispecific antibodies, which are based on heterodimeric Fc regions, are similar in shape and size to typical IgG, but the two Fab arms can recognize different Fc antigens.

The above described catenators may be fused to each of the two heavy chain C-termini or to each of the two light chain C-termini of the multispecific antibody, and thus, on a surface where target antigens are present, homodimers are formed between catenators to link multispecific antibodies to each other, thereby enhancing antigen-binding avidity of the antibody and improving the pharmacological function of the multispecific antibody.

Drug-Fc Fragment Conjugate

As used herein, the term “carrier” refers to a substance that binds to a drug to increase or decrease the physiological activity or increase in vivo stability of a physiologically active polypeptide. It is known in the art that the Fc region of an immunoglobulin can be used as a drug carrier.

The term “drug” means a substance that exhibits therapeutic activity when administered to humans or animals, and includes, but is not limited to, polypeptides, compounds, extracts, nucleic acids, and the like. Preferably the drug may be a polypeptide drug. Polypeptide drugs that can be linked with the Fc fragment may include hormones (e.g., intestinal hormones, growth hormone-releasing hormones, growth hormone-releasing peptides, etc.), interferons (e.g., interferon-α, -β, -γ, etc.), interleukins, growth factors, granulocyte colony stimulating factor (G-CSF), granulocyte-macrophage colony stimulating factor (GM-CSF), glucacon-like peptides (e.g., GLP-1, etc.), erythropoietin, enzymes, etc., but are not limited thereto. In addition, any derivative or analogs may be also included in the scope of the polypeptide drug of the present invention, as long as it has a function, structure, activity or stability substantially equal to or increased to that of the natural form of the polypeptide drug.

In addition to the polypeptide drugs, other drugs may also be linked to the Fc fragment. Non-limiting examples of these drugs include antibiotics selected from among derivatives and mixtures of tetracycline, minocycline, doxycycline, ofloxacin, levofloxacin, ciprofloxacin, clarithromycin, erythromycin, cefaclor, cefotaxime, imipenem, penicillin, gentamycin, streptomycin, vancomycin, and the like; anticancer agents selected from among derivatives and mixtures of methotrexate, carboplatin, taxol, cisplatin, 5-fluorouracil, doxorubicin, etoposide, paclitaxel, camtotecin, cytosine arabinoside, and the like; anti-inflammatory agents selected from among derivatives and mixtures of indomethacin, ibuprofen, ketoprofen, piroxicam, probiprofen, diclofenac, and the like; antiviral agents selected from among derivatives and mixtures of acyclovir and robavin; and antibacterial agents selected from among derivatives and mixtures of ketoconazole, itraconazole, fluconazole, amphotericin B and griseofulvin.

In general, the Fc region is in the form of a dimer of a heavy chain fragment composed of heavy chain C_{H2} and C_{H3} domains, and may include a hinge portion in some cases. The Fc fragment herein may contain a portion or the all the heavy-chain constant region 1 (C_{H1}) and/or the light-chain constant region 1 (C_{L1}), except for the variable regions of the heavy and light chains. Further, it may be a region from which part of the amino acid sequence corresponding to C_{H2} and/or C_{H3} is removed. The Fc fragment herein may include a native amino acid sequence and sequence derivatives (mutants) thereof. An amino acid sequence derivative is a sequence that is different from the native amino acid sequence due to a deletion, an insertion, a non-conservative or conservative substitution or combinations thereof of one or more amino acid residues. In addition, the Fc fragment herein may be in the form of having native sugar chains, increased sugar chains compared to a native form or decreased sugar chains compared to the native form, or may be in a deglycosylated form.

The Fc fragment herein may be fused to a physiologically active polypeptide through a peptide bond, or may be linked to a physiologically active polypeptide through a peptidic linker or a non-peptidic linker, but is not limited thereto.

The Fc fragment herein may be homodimeric or heterodimeric. In general, homodimerization of the Fc fragment is induced by a non-covalent bond between the last domains of an antibody constant region (C_{H3} domain in for IgG) and a disulfide bond between hinge regions. Heterodimeric Fc fragment can be generated through engineering so as to have a bond where heterodimerization is preferred and homodimerization is not preferred or inhibited, via a specific non-covalent bond between the last domains of a constant region that greatly contribute to homodimerization of naturally occurring antibodies. For example, through gene manipulation, mutations are induced in C_{H3} domains of two different antibody heavy chains so that the two heavy chains are very similar in structure to naturally occurring antibodies, have a minimal deviation in sequence, and form a heterodimer. Examples of technologies related to this include a knob-into-hole technology available from Genentech, ZW1 from Zymeworks, HA-TF available from XENECORE Co., Ltd, SEEDbody available from EMD Serono, and the like, but are not limited thereto.

In the drug-Fc conjugate herein, the drug may be conjugated to either or both of the N-terminal ends of the two copies of the Fc region.

The above described catenators may be fused to each of the two C-termini of the Fc included in drug-Fc conjugate, and thus, on a surface where target antigens are present, homodimers are formed between catenators to link drug-Fc conjugates to each other, thereby enhancing antigen-binding avidity of an antibody and improving the pharmacological function of the drug-Fc conjugate (FIG. 1E).

In particular, if the drug-Fc fragment conjugate herein is based on the heterodimeric Fc region, the catenators fused to the C-terminus of each of the two copies of the Fc region may be identical or different polypeptides.

Therapeutic Antibody

The term “therapeutic antibody” refers to an antibody that is used in the treatment of disease. A therapeutic antibody may have various mechanisms of action. A therapeutic antibody may bind and neutralize the normal function of a target. For example, a monoclonal antibody that blocks the activity of the protein needed for the survival of a cancer cell causes the cell's death. Another therapeutic monoclonal antibody may bind and activate the normal function of a target. For example, a monoclonal antibody can bind to a protein on a cell and trigger an apoptosis signal. Finally, if a monoclonal antibody binds to a target expressed only on diseased tissue, conjugation of a toxic payload (effective agent), such as a chemotherapeutic or radioactive agent, to the monoclonal antibody can create an agent for specific delivery of the toxic payload to the diseased tissue, reducing harm to healthy tissue.

The therapeutic antibody may be an antibody used for the treatment of various diseases, such as cancer (e.g., hematological cancer and solid cancer), immune disease, neurological disease, vascular disease, or infectious disease. Antigens for these antibodies may include tumor antigens such as tumor-associated antigen (TAA), tumor-specific antigen (TSA) or tumor-derived neoantigen; antigens of infectious agents, such as antigens derived from viruses, bacteria, parasites, or fungi; autoantigens known or suspected to induce autoimmunity; or a peptide derived from an allergen known or suspected to cause allergy, but is not limited thereto.

For example, such an antibody may be an antibody against ALK, adhesion related kinase receptor (e.g., Ax1), ERBB receptors (e.g., EGFR, ERBB2, ERBB3, ERBB4), erythropoietin-producing hepatocellular (EPH) receptors (e.g., EphA1, EphA2, EphA3, EphA4, EphA5, EphA6, EphA7, EphA8, EphB1, EphB2, EphB3, EphB4, EphB5, EphB6), fibroblast growth factor (FGF) receptors (e.g., FGFR1, FGFR2, FGFR3, FGFR4, FGFR5), Fgr, IGF1R, insulin receptors, LTK, M-CSFR, MUSK, platelet-derived growth factor (PDGF) receptors (e.g., PDGFR-A, PDGFR-B), RET, ROR1, ROR2, ROS, RYK, vascular endothelial growth factor (VEGF) receptors (e.g., VEGFR1/FLT1, VEGFR2/FLK1, VEGF3), tyrosine kinase with immunoglobulin-like and EGF-like domains (TIE) receptors (e.g., TIE-1, TIE-2/TEK), Tec, TYRO10, insulin-like growth factor (IGF) receptors (e.g., INS-R, IGF-IR, IR-R), Discoidin Domain (DD) receptors (e.g., DDR1, DDR2), receptor for c-Met (MET), recepteur d'origine nantais (RON, also known as macrophage stimulating 1 receptor), Flt3 (fins-related tyrosine kinase 3), colony stimulating factor 1 (CSF 1) receptor, receptor for c-kit (KIT or SCFR), insulin receptor related (IRR) receptors, CD19, CD20, HLA-DR, CD33, CD52, G250, GD3, PSMA, CD56, CEA, Lewis Y antigen, or IL-6 receptor, but is not limited thereto.

As for the therapeutic antibody herein, the features for the antibody as described above may be applied. The above described catenators may be fused to each of the two heavy chain C-termini or to each of the two light chain C-termini of the antibody or fragment thereof, and thus, on a surface where target antigens are present, homodimers are formed between catenators to link therapeutic antibodies to each other, thereby enhancing antigen-binding avidity of an antibody and improving the pharmacological function of the therapeutic antibody.

The therapeutic antibody herein may also be an antibody against a viral antigen. In a virus having multiple copies of the target antigen on its surface, the antibody-catenator may enhance antigen-binding avidity of an antibody to the target antigen on the surface of the virus, thereby greatly improving virus neutralizing activity.

Diagnostic Antibody

The term “diagnostic antibody” refers to an antibody that is used as a diagnostic reagent for a disease. The diagnostic antibody may bind to a target that is specifically associated with, or shows increased expression in, a particular disease. The diagnostic antibody may be used, for example, to detect a target in a biological sample from a patient, or in diagnostic imaging of disease sites, such as tumors, in a patient.

As for the diagnostic antibody herein, the features for the antibody as described above may be applied. The above described catenators may be fused to each of the two heavy chain C-termini or to each of the two light chain C-termini of the antibody or fragment thereof, and thus, on a surface where target antigens are present, homodimers are formed between catenators to link diagnostic antibodies to each other, thereby enhancing antigen-binding avidity of an antibody and facilitating detection of target biomarkers in biological samples.

In particular, the technology of the present disclosure can be useful when a very small number of biomarkers are present on target cells and thus a detection antibody with particularly high avidity is required to detect them.

In another example, the technology of the present disclosure may be applied to a secondary antibody of a sandwich type biosensor. The term “sandwich assay” refers to an immunological assay using two or more antibodies that bind to different sites on an antigen. For example, the assay typically includes a capture antibody and a detection antibody. As used herein, the term “capture antibody” refers to an antibody immobilized to a support so as to allow the antigen to bind to the support. When treating a biological sample containing a target biomarker (antigen), the biomarker (antigen) binds to the capture antibody to form an antigen-antibody complex. Then, when a secondary antibody that binds to another site of the biomarker (antigen) is added, it reacts with the antigen of the antigen-antibody complex, so that the presence or absence of the biomarker (antigen) in the sample can be easily detected.

The secondary antibody may be associated with one or more detectable labels to facilitate detection and/or quantification of the bound antigen. Such labels may include, but are not limited to, fluorescent substances, biotin moieties and/or enzymes. For example, enzymes may include peroxidase such as horseradish peroxidase (HRP), alkaline phosphatase, etc., fluorescent substances may include FITC, RITC, etc. In addition, radioactive isotope labels, latex bead labels, colloid labels, biotin labels, etc. may be used, but are not limited thereto.

The above described catenators may be fused to each of the two heavy chain C-termini or to each of the two light chain C-termini of the secondary antibody of a sandwich type biosensor, and thus, on a surface where target antigens are present, homodimers are formed between catenators to the antibodies to each other, thereby enhancing antigen-binding avidity of an antibody and greatly improving the detection sensitivity of the biomarker (antigen) in the sample.

Linker

Meanwhile, in another example of the conjugate of the present disclosure, the catenator polypeptide may be linked to an antibody or a fragment thereof through a linker.

The term “linker” refers to a group of atoms or a molecule that connects or couples or binds two or more components together. The linker may be a water-soluble and/or flexible linker. The linker may have additional functions such as increasing or decreasing water solubility, increasing the distance between two components to be linked to provide flexibility or increase stability, but it is preferable not to induce immunogenicity or affect the activity of the conjugate.

In a preferred embodiment, the linker may be a peptide linker, for example, having a length of 1 to 10 amino acids, or 2 to 10 amino acids, specifically, 1, 2, 3, 4, 5, 6, 7, 8, 9, or 10 amino acids, or having a length of more than 10 amino acids, specifically, 11, 12, 13, 14, 15, 16, 17, 18, 19, or 20 amino acids, but is not limited thereto. In one embodiment, the peptide linker may be composed of neutral amino acids (e.g., Gly, Ser, Ala, Thr or a combination of these four amino acids). For example, it may be (GS)ₙ, (G₂S)ₙ, (G₃S)ₙ, (G₄S)ₙ, Gₙ, LE, SSGG or GGGGSGGGGG (G is Gly, S is Ser, L is Leu, E is Glu, and n is an integer of at least 1), specifically, GS, GGGGS, LE, SSGG, GG, GGGGG or GGGGSGGGGG, but is not limited thereto.

Preparing Method

The conjugate of the present application may preferably be obtained by expression and purification by a recombinant method, but is not limited thereto. Accordingly, for the expression and purification of the conjugate, the present invention also provides an expression vector comprising a nucleic acid molecule encoding the conjugate, and a cell transformed therewith.

In another embodiment, provided is an expression vector comprising the nucleic acid molecule.

The term “expression vector” refers to a nucleic acid construct operably linked to express a gene insert encoding a target protein. In one embodiment, the expression vector may be a linear or circular, single-stranded or double stranded DNA, cDNA, or RNA encoding two or more target proteins. The expression vector may be used to transform or transfect a host, but is not limited thereto, and the gene construct itself may be transcribed and/or translated in vitro.

The term “operably linked” refers to a functional linkage between nucleic acid sequences. For example, a coding sequence (e.g., a sequence encoding a target protein) may be operably linked to appropriate control elements to allow replication, transcription, and/or translation thereof. For example, the coding sequence is operably linked to a promoter when the promoter directs transcription of the coding sequence. The control elements need not be contiguous with the coding sequence, as long as they function correctly. For example, intervening untranslated yet transcribed sequences may be present between the promoter sequence and the coding sequence, and the promoter may still be considered “operably linked” to the coding sequence.

Respective components in the expression vector must be operably linked to each other, and linkage of these component sequences may be performed by ligation at a convenient restriction enzyme site, and when such a site does not exist, the ligation may be performed using a synthetic oligonucleotide adapter or linker according to a common method.

The expression vector may comprise transcriptional and translational expression control sequences that allow the gene to be expressed in a selected host. The expression control sequence may comprise a promoter for performing transcription, a random operator sequence for controlling such transcription, and/or a sequence for controlling the termination of transcription and translation. Start codons and stop codons are generally considered as part of a nucleic acid sequence that encodes a target protein. It is necessary that they are functional in an individual to whom the gene construct is administered. The start codons and stop codons must be in frame with the coding sequence.

For example, promoter refers to a DNA sequence site to which transcriptional regulators bind. With respect to the objects of the present invention, a promoter capable of inducing strong and stable gene expression may be used to increase a gene expression rate.

The promoter may be constitutive or inducible. The promoter may be exemplified by, but is not limited to, adenovirus early and late promoters, simian virus 40 (SV40), mouse mammary tumor virus (MMTV) promoter, HIV long terminal repeat (LTR) promoter, Moloney virus, cytomegalovirus (CMV) promoter, Epstein Barr virus (EBV) promoter, Rous sarcoma virus (RSV) promoter, RNA polymerase±promoter, T3 and T7 promoters, major operator and promoter regions of phage lambda, etc.

In addition, the expression vector may appropriately comprise an adapter or a linker, an enhancer, a selectable marker (e.g., antibiotic resistance marker), a replication unit, a polyA sequence, a tag for purification or other sequences of construction and induction known to regulate gene expression of prokaryotic or eukaryotic cells or viruses thereof and various combinations thereof, etc. Various types of vectors such as plasmids, viral vectors, bacteriophage vectors, cosmid vectors, etc. may be used.

Still another embodiment relates to a cell transformed with the expression vector.

With respect to the transformed cell, any host cell known in the pertinent art with the ability to stably and continuously clone and express the expression vector can be used. The prokaryotic cell available as a host includes E. coli, such as E. coli JM109, E. coli BL21, E. coli RR1, E. coli LE392, E. coli B, E. coli X 1776, or E. coli W3110, Bacillus sp. strain, such as Bacillus subtillus or Bacillus thuringiensis, and intestinal bacterial strains, such as Salmonella typhimurium, Serratia marcescens, various Pseudomonas sp., and the like. The eukaryotic cell available as a host includes Saccharomyces cerevisiae, an insect cell, a plant cell and an animal cell, such as CHO (Chinese hamster ovary), W138, BHK, COS-7, 293, HepG2, 3T3, RIN, and MDCK cell line, and the like, but not limited thereto.

Introduction of the expression vector into cells may be performed by using appropriate standard techniques as known in the art, for example, electroporation, electroinjection, microinjection, calcium phosphate co-precipitation, a calcium chloride/rubidium chloride method, retroviral infection, DEAE-dextran, a cationic liposome method, polyethylene glycol-mediated uptake, gene guns, etc., but is not limited thereto. At this time, the vector may be introduced in a linearized form by digestion of a circular construct with appropriate restriction enzymes.

A method of selecting the transformed host cell may be easily carried out using a phenotype expressed by a selection marker according to a method well known in the art. For example, when the selection marker is a specific antibiotic resistance gene, the transformant may be easily selected by culturing the transformant in a medium containing the antibiotic.

The transformed cell may be cultured using various a known method in the art. For example, a transformed cell may be inoculated into a culture medium and cultured therein, and isopropyl β-D-1-thiogalactopyranoside (IPTG) may be added to the medium to induce protein expression at a time when the density of the cell reaches a certain level. After culturing the cells, conjugate expressed within the cell or secreted to the culture medium may be collected.

The conjugate expressed inside the cell or secreted into the medium may be obtained in a purified form by using one of various known purification methods in the art, preferably, it may be purified through affinity chromatography using affinity tags. For example, if the conjugate is fused to glutathione-S-transferase (GST), the conjugate may easily be obtained using a glutathione-binding resin column, and if fused to poly-Histidine-tag, the conjugate may easily be obtained using IMAC (immobilized Metal Affinity Chromatography).

Composition and Method

In other aspects, the present invention provides a composition for enhancing antigen-binding avidity of an antibody, the composition comprising the conjugate as described above, which is characterized in that, on the surface where target antigens to which the antibody included in the conjugate specifically binds are present, pairs of the conjugate-antigen complexes adjacent to each other are catenated by intermolecular dimerization between catenator polypeptides, thereby suppressing dissociation of the antibody.

In other aspects, the present invention provides a method for enhancing antigen-binding avidity of an antibody, comprising treating the conjugate as described above on the surface where target antigens to which the antibody specifically binds are present, which is characterized in that, on the surface, pairs of the conjugate-antigen complexes adjacent to each other are catenated by intermolecular dimerization between catenator polypeptides, thereby suppressing dissociation of the antibody.

The term “antigen-antibody binding ability” is intended to include antigen-binding avidity of an antibody. The antigen-binding avidity of an antibody refers to the sum of binding interactions between multiple antigen determinants present in one antigen and multiple binding sites present in one antibody during antigen-antibody binding, or the overall strength of antigen-antibody interaction. The antigen-antibody binding ability can be measured using any method known in the art. Such methods include, for example, fluorescence activated cell sorting (FACS), surface plasmon resonance (e.g., Biacore, ProteOn), biolayer interferometry (BLI, e.g. Octet), kinetics exclusion assay (e.g. KinExA), separable beads (e.g., magnetic beads), antigen panning, and/or ELISA.

When the antibody herein is a therapeutic antibody, administration of the composition may prevent a disease, or inhibit, stop or delay the onset or progression of a disease state, or ameliorate or beneficially alter symptoms.

The term “effective amount” refers to an amount sufficient to achieve the desired result, e.g., an amount effective to treat or prevent a disease, when administered to subjects, including humans. The effective amount may vary depending on various factors such as a formulation method, administration mode, a patient's age, body weight, sex, disease severity, diet, administration time, administration route, excretion rate, and response sensitivity. Administration dosage or therapeutic regimen may be adjusted to provide an optimal therapeutic response as will be understood by those skilled in the art.

The composition of the present disclosure may be provided together with one or more additives selected from the group consisting of pharmaceutically acceptable carriers, diluents, and excipients.

The pharmaceutically acceptable carrier, which is commonly used in the formulation of antibody, may be one or more selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc., but is not limited thereto. The composition may further include one or more selected from the group consisting of diluents, excipients, lubricants, wetting agents, sweeteners, flavoring agents, emulsifiers, suspending agents, preservatives, etc., which are commonly used in the preparation of pharmaceutical compositions, in addition to the above components. The pharmaceutically acceptable carriers and formulations suitable for the present disclosure, including those exemplified above, are described in detail in the literature [Remington's Pharmaceutical Sciences, latest edition].

The composition may be administered orally or parenterally. When administered parenterally, intravenous injection, subcutaneous injection, intramuscular injection, intraperitoneal injection, endothelial administration, topical administration, intranasal administration, intraocular administration, intrathecal administration, intrathecal administration, intracranial administration, and intrastriatal administration may be used.

In some embodiments, the composition is provided as a sterile liquid preparation, for example, as an isotonic aqueous solution, a suspension, an emulsion, a dispersion, or a viscous composition, which, in some aspects, may be buffered to a selected pH. Liquid preparations are normally easier to prepare than gels, other viscous compositions, and solid compositions. In addition, liquid compositions are particularly convenient to administer by injection. Viscous compositions, on the other hand, may be formulated within the appropriate viscosity range to provide longer contact periods with a specific tissue. Liquid or viscous compositions may include a carrier which may be a solvent or dispersion medium containing, for example, water, saline, phosphate buffered saline, polyol (e.g., glycerol, propylene glycol, liquid polyethylene glycol) and appropriate mixtures thereof.

Sterile injectable solutions may be prepared by incorporating the binding molecule in a solvent, such as in admixture with a suitable carrier, diluent, or excipient such as sterile water, physiological saline, glucose, dextrose, or the like. The compositions may also be lyophilized. The compositions may include auxiliary substances such as wetting, dispersing, or emulsifying agents (e.g., methylcellulose), pH buffering agents, gelling or viscosity enhancing additives, preservatives, flavoring agents, colors, etc., depending upon the route of administration and the desired preparation.

Various additives which enhance stability and sterility of the compositions, including antimicrobial preservatives, antioxidants, chelating agents, and buffers, may be added. Prevention of microbial actions may be ensured by various antibacterial and antifungal agents, for example, parabens, chlorobutanol, phenol, sorbic acid, etc. Prolonged absorption of the injectable pharmaceutical form may be brought about by the use of agents delaying absorption, for example, aluminum monostearate and gelatin.

### EXAMPLES

Hereinafter, the present invention will be described in more detail with reference to the following examples. However, these examples are only for illustrating the present invention, and the scope of the present invention is not limited by these examples.

### Example 1. MCMC (Markov Chain Monte-Carlo) Simulation

Simulation runs were carried out in the three steps stated below with specification of the target surface, K_{D} (for antibody-antigen interaction), (K_{D})_{catenator} (for catenator-catenator interaction) and ƒ(d) (effective local concentration of the catenator). A protein in which an antibody and a catenator are fused were named ‘an antibody-catenator (^{cat}Ab)’. In all simulations, the number of ^{cat}Ab was far more than that of the binding sites, and therefore, the concentration of free ^{cat}Ab was assumed to be the same as that of totoal ^{cat}Ab (free ^{cat}Ab+antigen-bound ^{cat}Ab). The definition and values of the parameters used in the presented simulations are shown in Table 2.

TABLE 2

| | | |
|---|---|---|
| Parameters | Description | Values |

Specification of ^{cat}Ab

| K_{D} | Dissociation constant of antibody | 10 | nM |
|---|---|---|---|
| (K_{D})_{catenator} | Dissociation constant of catenator | 10 nM-10 mM | |
| [^{cat}Ab] | Antibody concentration | 1 | nM |
| l | Length of the flexible linker | 6 | nm |
| c | Length of the catenator | 2 | nm |
| L | Reach length (l + c/2) | 7 | nm |

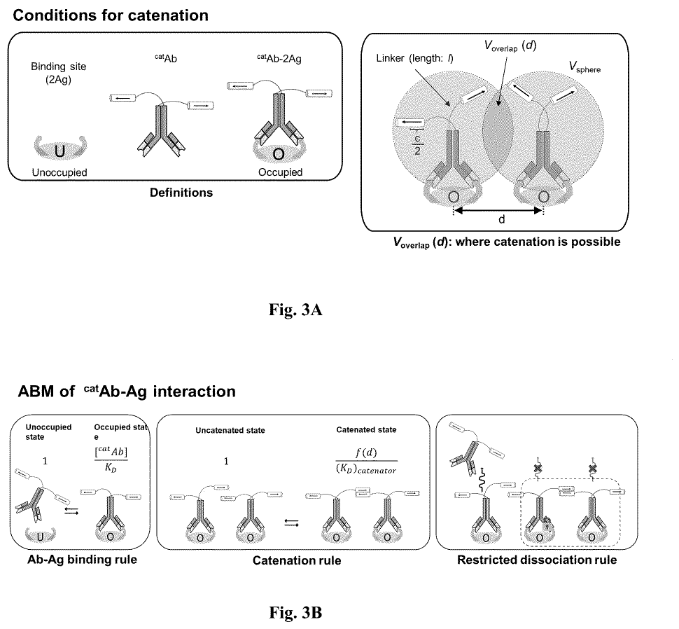

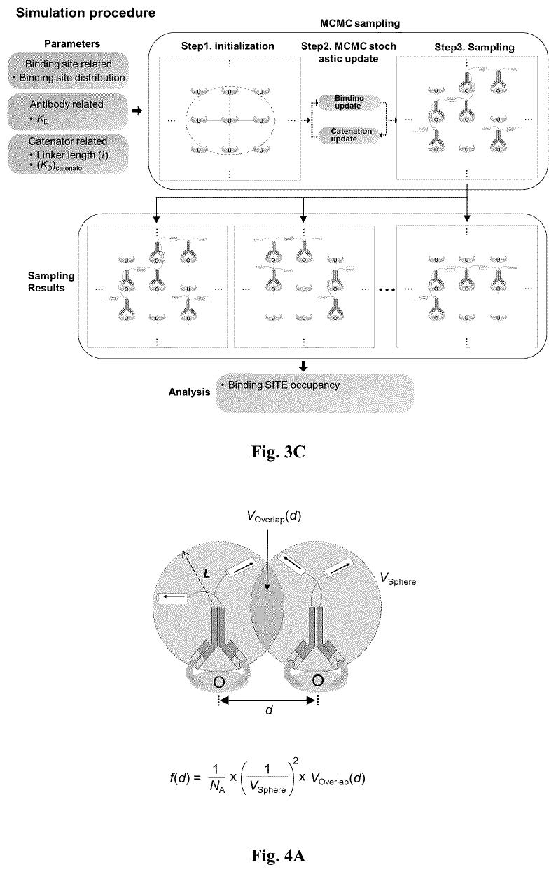

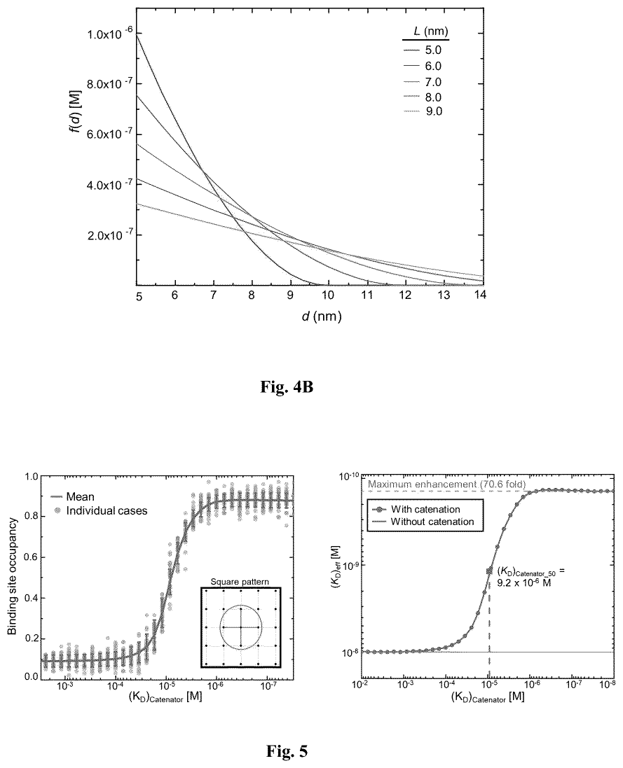

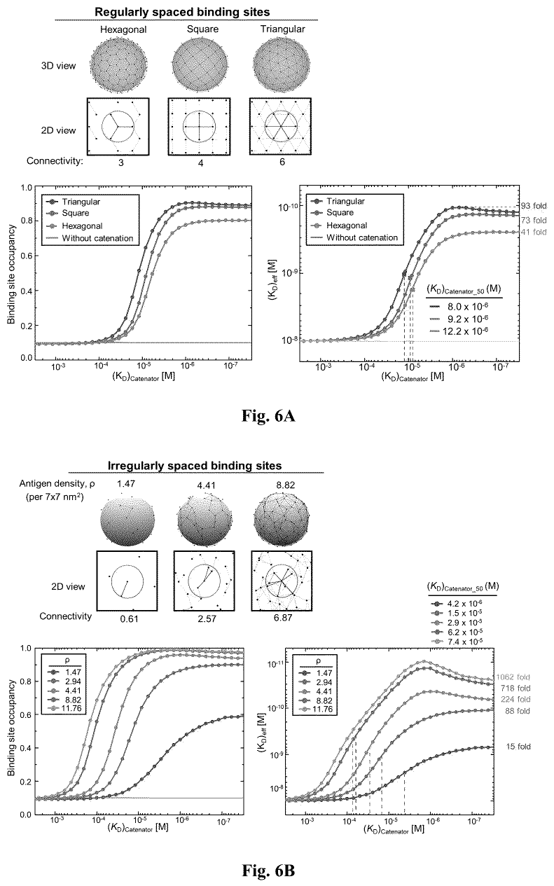

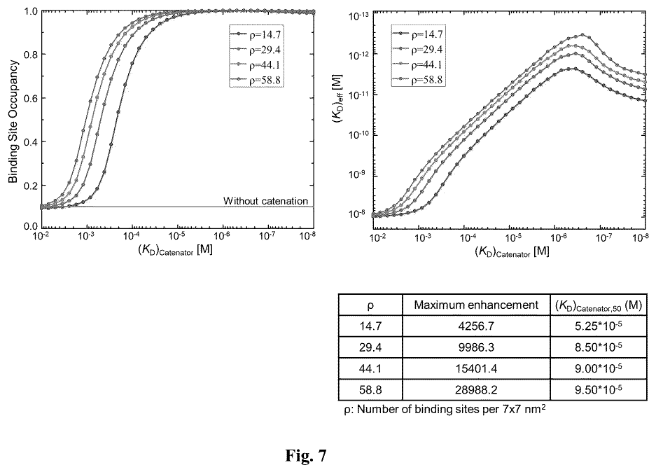

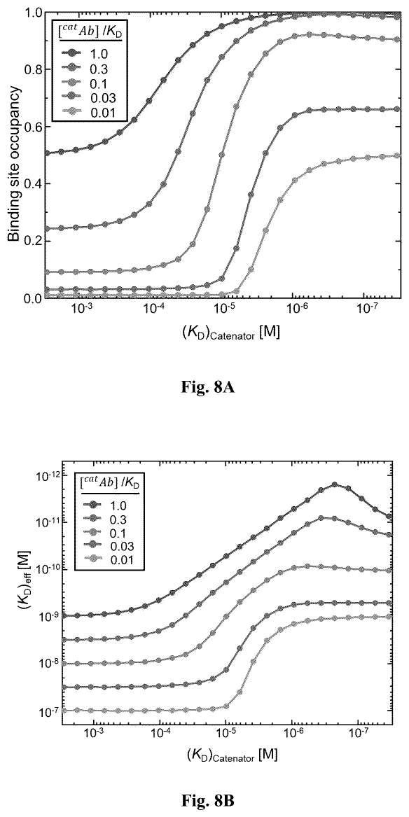

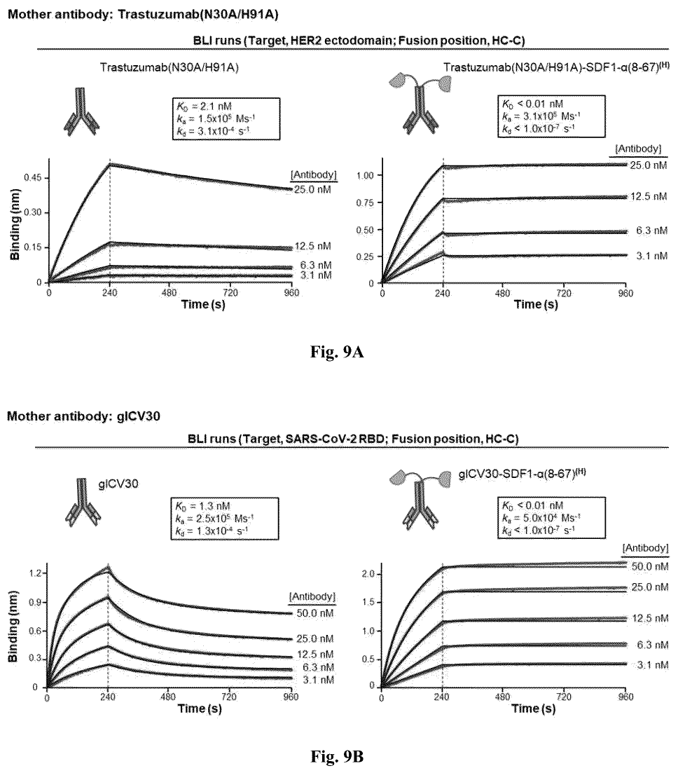

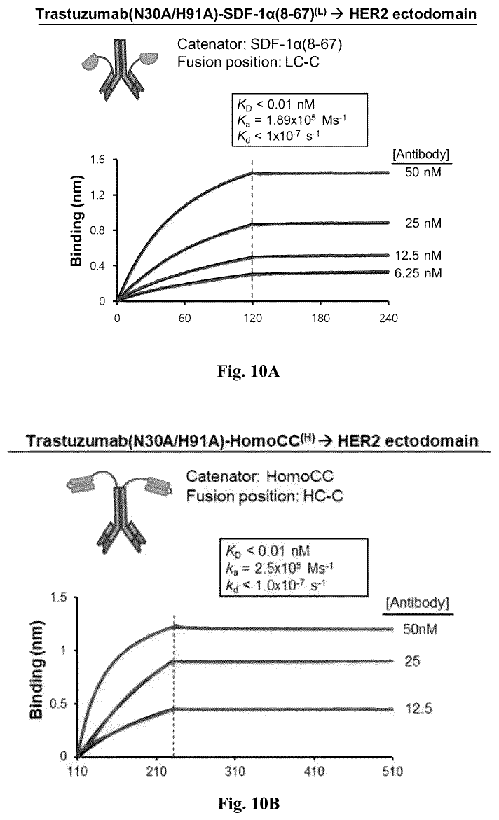

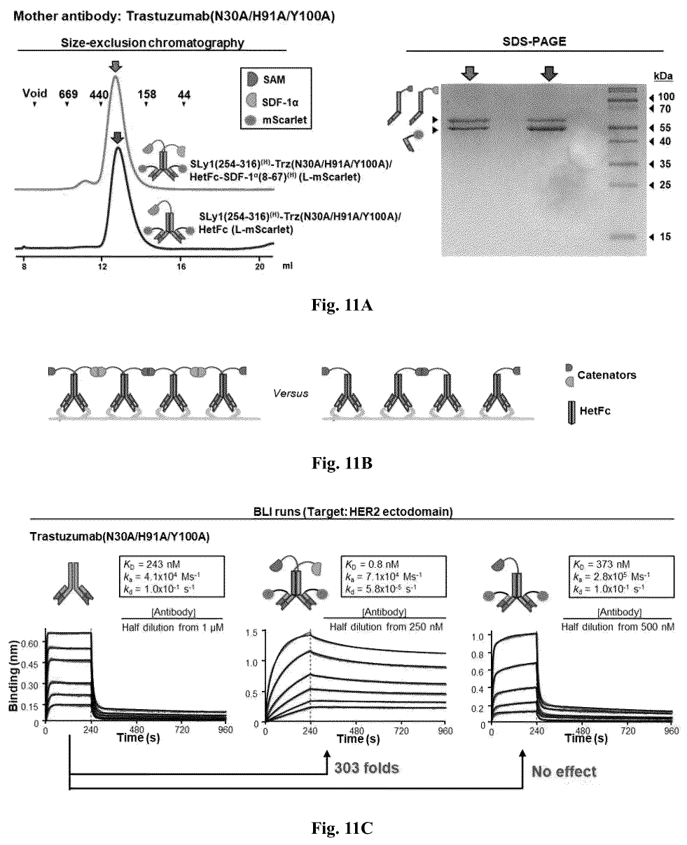

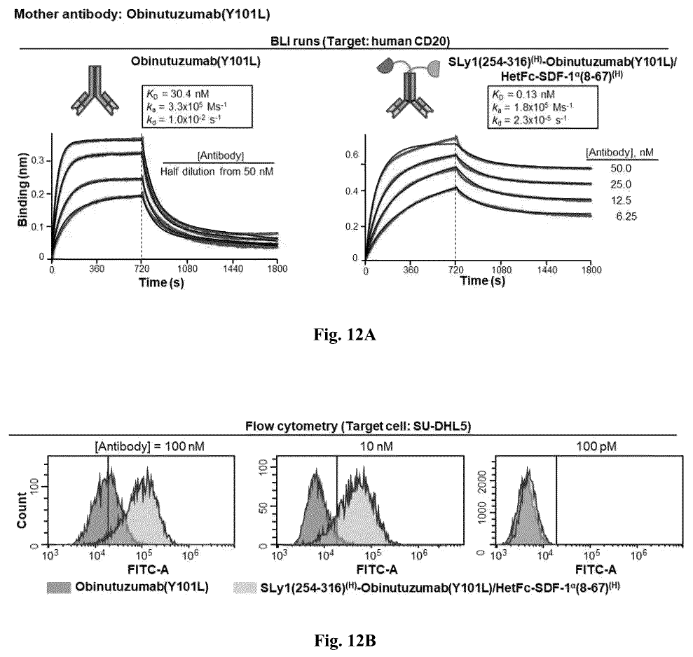

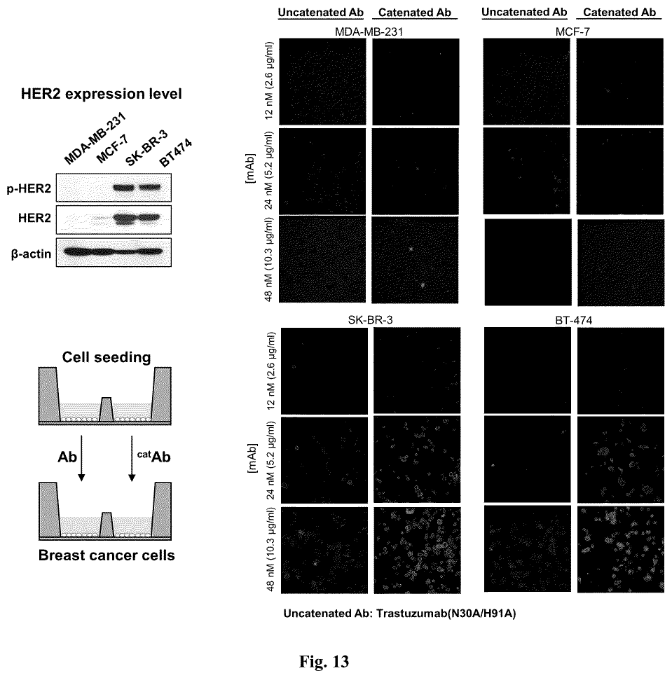

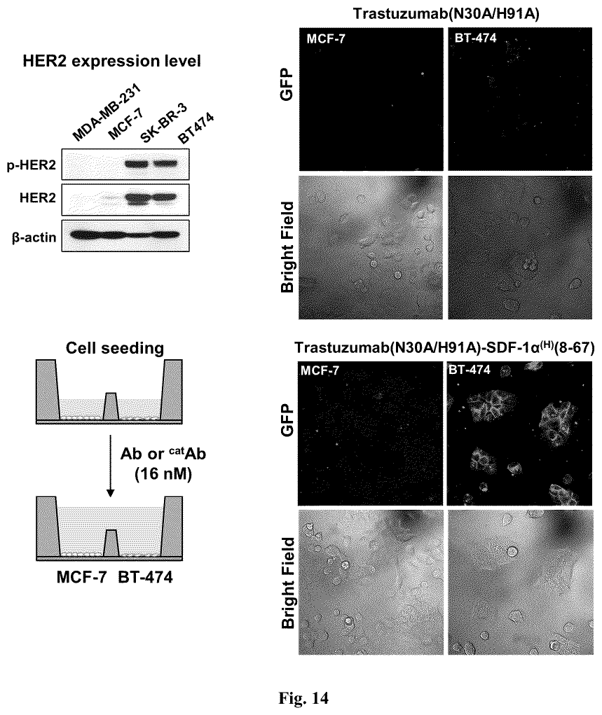

Specification of target surface

| Nₜₒₜₐₗ_{_{—}}_{binding}_{_{—}}ₛᵢₜₑₛ | Number of antibody-binding sites | 98-102 | |
|---|---|---|---|
| (in FIG. 3 and FIG. 4) | | | |
| Connectivity number | Number of possible catenation | 3 | (Hexagonal) |
| (in FIG. 3 and FIG. 4) | | 4 | (Square) |
| | | 6 | (Triangular) |
| d (in FIG. 3 and FIG. 4) | Distance between adjacent binding sites | 12 | nm |
| L_{surface} (in FIG. 4) | Surface area of the target surface | 40 | nm² |

| | | |
|---|---|---|
| Binding site density | Surface density of the binding sites | 0.5-4.0 |
| (in FIG. 4) | | (per 12 × 12 nm²) |

Specification of Simulation

| | | |
|---|---|---|
| Updates/MCMC step | Number of updates in one MCMC step | 30,000-100,000 |
| Sampling size | Number of sampling for a parameter set | 1,024 |

Step 1. Initialization Step

1. A specified 3D target surface is implemented by assigning binding sites to specific locations on the surface.

2. Each binding site is set to be unoccupied.

Step 2. MCMC Stochastic Update Step

The following sub-steps (1-3) are iterated for sufficient times to ensure thermodynamic equilibration.

1. A random binding site BS₁ is chosen from the target surface.

2. The binding status of BS₁ is updated.

If BS₁ is unoccupied, its status is changed to the occupied status with the acceptance probability of

$$max ( 1 , \frac{[ {}^{cat} Ab ]}{{K}_{D}} ) .$$

If BS₁ is occupied, its status is changed to the unoccupied status with the acceptance probability of

$$max ( 1 , \frac{{K}_{D}}{[ {}^{cat} Ab ]} ) .$$

3. An occupied binding site BS₂ right next to BS₁ is picked on the target surface, and the catenation status of the pair (BS₁, BS₂) is updated.

If (BS₁, BS₂) is uncatenated, and if both BS₁ and BS₂ have an unengaged catenator, its status is changed to the catenation status with the acceptance probability of

$$max ( 1 , \frac{f ( d )}{{( {K}_{D} )}_{catenator}} ) .$$

Step 3. Sampling Step

1. It is stopped to update the target surface, and the final status are recorded.

2. The total number of occupied and unoccupied binding sites are counted.

The codes for the model system and simulations are available in MATLAB and available on Github (https://github.com/JinyeopSong/Antibody_ThermoCalc_JY). The detailed description is provided in Readme.

### Example 2. Preparation of Original Antibodies and Catenator-Fused Antibodies

2-1. Selection of Catenators

Three different homodimer-forming proteins were used as catenators. One is a polypeptide consisting of amino acid residues 8-67 of the human chemokine protein, stromal cell-derived factor-1a (SDF-1α), in which 7 amino acid residues having a signaling function are removed from the N-terminus of the full-length protein (Ryu et al., (2007) Crystal structure of recombinant human stromal cell-derived factor-1a. PROTEINS: Structure, Function, and Bioinformatics, 67(4), 1193-1197). It is small in size (Mr=8 kDa) and has a very low homodimerization affinity of 140 μM (Veldkamp et al., (2005) The monomer-dimer equilibrium of stromal cell-derived factor-1 (CXCL 12) is altered by pH, phosphate, sulfate, and heparin. Protein Science, 14(4), 1071-1081). Another is Sterile Alpha Motif domain (SAM) domain consisting of amino acid residues 254-316 of SH3 domain-containing protein expressed in lymphocytes 1 (SLy1). It is small in size (Mr=7.4 kDa) and weakly forms homodimers (K_{D}=117 μM) (Kukuk et al., (2019) Structure of the SLy1 SAM homodimer reveals a new interface for SAM domain self-association. Sci Rep. 9, 54). The other is a polypeptide named ‘homodimeric coiled-coil (HomoCC)’, a coiled-coil type protein designed de novo by the present inventors. HomoCC is small in size (Mr=8.7 kDa) and has a homodimerization affinity of 7.6 μM (FIG. 2).

2-2. Preparation of Antibody-Catenator (^{cat}Ab)

Eight antibody-catenators (^{cat}Ab) were prepared by combining 4 different antibodies (g1CV30, Trastuzumab(N30A/H91A), Trastuzumab (N30A/H91A/Y100A), Obinutuzumab(F101L)) with 3 catenators (Table 3). Four of these conjugates were prepared by fusing the same catenator to homodimeric Fc, and the other four conjugates were prepared by fusing SDF-1a (8-67) and SLy1 (254-316) to each C-terminus of the heavy chain of heterodimeric Fc (HetFc). In order to prepare HetFc, referring to the “knobs-in-holes” type HetFc (PDB: 5DI8), T366W was introduced to C_{H3} of one heavy chain and T366S, L358V, and Y407A mutations were introduced to CH3 of the other heavy chain. The reason for introducing mutations into the original Trastuzumab and the original Obinutuzumab was to artificially lower the antigen-binding ability of these antibodies.

TABLE 3

| | | | Fusion | | Linker |
|---|---|---|---|---|---|
| Antigen | Antibody | Antibody-catenator | position | Catenator | peptide |
| RBD | glCV30 | glCV30-SDF-1α(8-67)^{(H)} | HC-C | SDF-1α | GGGGSGG |
| | | | | (8-67) | GGS |
| RBD | glCV30 | SLy1(254-316)^{(H)}- | HC-C | SDF-1α | GGGGSGG |
| | | glCV30/HetFc-SDF-1α | | (8-67) | GGS |
| | | (8-67)^{(H)} | | | |
| HER2 | Trastuzumab | Trastuzumab(N30A/H91A)- | HC-C | SDF-1α | GGGGSGG |
| | (N30A/H91A) | SDF-1α(8-67)^{(H)} | | (8-67) | GGS |
| HER2 | Trastuzumab | Trastuzumab(N30A/H91A)- | LC-C | SDF-1α | GGGGSGG |
| | (N30A/H91A) | SDF-1α(8-67)^{(L)} | | (8-67) | GGS |
| HER2 | Trastuzumab | Trastuzumab(N30A/H91A)- | HC-C | HomoCC | GGGGSGG |
| | (N30A/H91A) | HomoCC^{(H)} | | | GGS |
| HER2 | Trastuzumab | SLy1(254-316)^{(H)}- | HC-C | Sly1 | GGGGSGG |
| | (N30A/H91A)/ | Trastuzumab(N30A/H91A)/ | | (254-316), | GGS |
| | HetFC | HetFc-SDF-1α(8-67)^{(H)} | | SDF-1α | |
| | | | | (8-67) | |
| HER2 | Trastuzumab | SLy1(254-316)^{(H)}- | HC-CLC-C | Sly1 | GGGGSGG |
| | (N30A/H91A/ | Trastuzumab(N30A/H91A/ | | (254-316), | GGS |
| | Y100A)/HetFc | Y100A)/HetFc-SDF-1α | | SDF-1α | |
| | | (8-67)^{(H)} (L-mScarlet) | | (8-67) | |
| CD20 | Obinutuzumab | SLy1(254-316)^{(H)}- | HC-C | SDF-1α | GGGGSGG |
| | (F101L) | Obinutuzumab(F101L)/ | | (8-67) | GGS |
| | | HetFc-SDF-1α(8-67)^{(H)} | | | |

RBD: Receptor-binding domain of the SARS-COV-2 spike protein
HER2: Human ERBB2
CD20: Cluster of differentiation 20
Trastuzumab(N30A/H91A): Trastuzumab containing the N30A and H91A mutations (Slaga et al., (2018) Sci Transl Med 10(463))
Trastuzumab(N30A/H91A/Y100A)/HetFc: Trastuzumab (N30A/H91A/Y100A) containing Heterodimeric Fc
glCV30: germ-line antibody against the SARS-COV-2 RBD (Hurlburt, et al. (2020) Nat Commun 11, 5413)
Obinutuzumab(F101L): Obinutuzumab containing F101L mutation
^{(H)}Fusion to heavy chain;
^{(L)}Fusion to light chain
SDF-1α(8-67): Stroma cell-derived factor-la, residues 8-67
HomoCC: De novo designed homodimeric coiled-coil
SLy1(254-316): SH3 domain-containing protein expressed in lymphocytes 1, residues 254-316 (=SAM domain; Sterile Alpha Motif domain) (Kukuk et al., (2019) Sci Rep. 9, 54)
HC-C: C-terminus of Heavy chain;
LC-C: C-terminus of Light chain
L-mScarlet: mScarlet fused to light chain
G: glycine;
S: Serine

Each DNA fragment encoding heavy chain variable regions (V_{H}) and light chain variable regions (V_{L}) of glCV30 was synthesized (IDT) and cloned into the pCEP4 vector (Invitrogen). DNA fragments of C_{H1}-C_{H2}—C_{H3} of the gamma heavy chain and C_{L} of the kappa-type light chain were inserted into the V_{H} and V_{L}, and the resulting vectors were named glCV30 Hc and glCV30 Lc, respectively. DNA fragment encoding SDF-1α(8-67) was synthesized (IDT) and cloned into the glCV30 Hc next to C_{H3} of glCV30 with (Gly-Gly-Gly-Gly-Ser) 2 linker sequence glCV30-SDF-1α(8-67) Hc. For antibody production, the three vectors were amplified using the NucleoBond Xtra Midi kit (Macherey-Nagel), and a combination of the glCV30 Hc and glCV30 Lc vectors or a combination of the glCV30-SDF-1α He and glCV30 Lc vectors were introduced into the ExpiCHO-S cells (Gibco). The transfected cells were grown in the ExpiCHO expression medium (Gibco) for ten days post-transfection. Supernatants were collected by centrifugation at 4° C., filtered through 0.45 μm filters (Millipore), diluted by addition of a binding buffer (150 mM NaCl, 20 mM Na₂HPO₄, pH 7.0) to a 1:1 ratio, loaded onto an open column containing Protein A resin (Sino Biological), and eluted with an elution buffer (0.1 M glycine, pH 3.0). The eluent was immediately neutralized by a neutralizing buffer (1M Tris-HCl, pH 8.5), and the antibodies were further purified using a HiLoad 26/60 Superdex 200 gel-filtration column (Cytiva) equilibrated with a buffer solution containing 20 mM Tris-HCl (pH 7.5) and 150 mM NaCl.

To prepare CV30, the CV30 Hc vector was cloned by introducing F27V and T28I mutations to glCV30 Hc. Except for the use of CV30 Hc and glCV30 Lc for transfection, the protein production and purification processes are generally the same as those used for glCV30.

To prepare original Trastuzumab (‘Trastuzumab’) and Trastuzumab conjugated with SDF-1a (8-67) or HomoCC or SLy1 (254-316) (′ Trastuzumab-SDF-1α(8-67)^{(H)}′, ‘Trastuzumab-SDF-1α(8-67)^{(L)}’, ‘Trastuzumab-HomoCC^{(H)}’, ‘Trastuzumab-Sly1(254-316)^{(H)}’), each DNA fragment encoding heavy chain variable region (VH) and light chain variable region (VL) of Trastuzumab, HomoCC, and Sly1 (254-316) was synthesized (IDT). N30A/H91A mutations were introduced into the light chain of each Trastuzumab. The cloning, protein production, and purification processes are generally the same as those used for glCV30 and glCV30-SDF-1a (8-67). However, in the case of Trastuzumab(N30A/H91A)-SDF-1α(8-67)^{(L)}, SDF-1α(8-67) was cloned together with (Gly-Gly-Gly-Gly-Ser) 2 linker at the terminal of Trastuzumab Lc. When HomoCC was used as a catenator, the linker sequence Gly-Gly-Gly-Ser was used.

To prepare Trastuzumab (SLy1(254-316)^{(H)}-Trastuzumab(N30A/H91A)/HetFc-SDF-1α(8-67)^{(H)} in which SLy1(254-316) and SDF-1α(8-67) were conjugated to Trastuzumab(N30A/H91A)/HetFc, DNA fragment encoding heavy chain variable region (VH) and light chain variable region (VL) of Trastuzumab(N30A/H91A) was synthesized (IDT). The linker sequence connecting the catenators was (Gly-Gly-Gly-Gly-Ser) 2. A 6x(His) tag was conjugated to the C-terminus of SLy1 (254-316) and maltose binding protein (MBP) was conjugated to the C-terminus of SDF-1α (8-67). Other cloning and protein production processes are generally the same as those used for glCV30. Ni-NTA resin and amylose resin were used for protein purification, and MBP was cut out from the antibody using TEV protein cleavage enzyme.

The cloning, protein production, and purification processes for SLy1(254-316)^{(H)}-Trastuzumab(N30A/H91A/Y100A)/HetFc-SDF-1α(8-67)^{(H)} and SLy1(254-316)^{(H)}-Obinutuzumab(F101L)/HetFc-SDF-1 α(8-67)^{(H)} are generally the same as those used for SLy1(254-316)^{(H)}-Trastuzumab(N30A/H91A)/HetFc-SDF-1α(8-67)^{(H)}.

### Example 3. Bio-Layer Interferometry (BLI)

BLI experiments were performed to measure dissociation constants using an Octet R8 (Sartorius). Biotinylated SARS-CoV-2 RBD (Acrobio system) or biotinylated Her2/ERBB2 (Sino Biological) was loaded to a streptavidin biosensor tip (Sartorius) for 120 s. A baseline was determined by incubating the sensor with Kinetics Buffer (Sartorius) for 60 s. Antibody samples at different concentrations went through the association phase for 240 s and the dissociation phase for 720 s. All reactions were carried out in the Kinetics Buffer (Satorius). The binding kinetics were analyzed using the Octet DataAnalysis 10.0 software (Sartorius) to deduce the kinetic parameters.

### Example 4. Cell Line Analysis

The HER expression level of the breast cancer cell lines, MDA-MB-231(ATCC), MCF-7(ATCC), SK-BR-3(ATCC) and BT474(ATCC), was confirmed by Western blotting. Each cell line was seeded in two separate wells, and Trastuzumab(N30A/H91A) and Trastuzumab(N30A/H91A)-SDF-1α(8-67)^{(H)}, in which mScarlet or GFP fluorescent protein is fused to the light chain C-terminus, were added at different concentrations and analyzed by confocal microscopy after 30 minutes. Since a lymphoma cell line, su-DHL-5 (DSMZ) is a floating cell, the cell binding activity of the antibody was analyzed by fluorescence assisted cell sorting (FACS) method instead of confocal microscopy.

### Example 5. Virus Neutralizing Activity Analysis

To prepare a vesicular stomatitis virus (VSV) pseudotyped with the SARS-CoV-2 spike protein of the Wuhan-Hu-1 strain, HEK293T cells, which were previously plated overnight at 3×10⁶ cells in 10 cm dishes, were transfected with 15 μg plasmid encoding the spike protein of SARS-CoV-2 with 18 residues deleted in the cytoplasmic tail using calcium phosphate. 24 hours after transfection, cells expressing the spike protein were infected with a recombinant VSV in which the G gene was replaced with a luciferase gene (rVSV-ΔG-Luc) for 1 hour. Cells were washed three times with Dulbecco's phosphate-buffered saline, and 7-10 mL of the same medium containing 10% FBS was added. Culture medium was harvested 24-48 hours after infection, filtered with a 0.45 μm filter, and VSV pseudotyped with the SARS-CoV-2 spike protein of Wuhan-Hu-1 strain was obtained (rVSV-ΔG-Luc). It was stored at −80° C. for neutralization assay.

Each of the three antibodies (CV30, glCV30 and glCV30-SDF-1α(8-67)) was serially diluted and added to rVSV-ΔG-Luc containing SARS-CoV-2 spike protein from Wuhan-Hu-1 strain, and the mixture was incubated with HEK293T-hACE2 cells and luciferase activity was measured and IC₅₀ values were obtained.

RESULTS

1. The Concept of Antibody Catenation on a Target Surface

This concept was derived from (i) the unique dimeric structure of the IgG-type antibody and (ii) a proximity effect that potentially takes place on a target cell surface. In the structure of IgG, the crystallizable fragment (Fc) is composed of two copies of the constant regions of the heavy chain (C_{H2} and C_{H3}) forming a homodimer, in which the two C-termini are ˜23 Å apart and point away from each other (FIG. 1A, Left). This structural feature indicated that a homodimer-forming protein genetically fused to the C-terminus can be prevented from forming a homodimer intramolecularly by controlling the length of the connecting linker or its homodimerization affinity. Instead, the fusion protein can form a homodimer intermolecularly, and then such a homodimerization could result in a catenation of the antibody molecules (FIG. 1A, Right). We designate the fusion protein between an antibody and a homodimeric protein as antibody-catenator (^{cat}Ab). A proximity effect for ^{cat}Ab is expected on a target surface where multiple copies of target antigen are present, because the local concentration of ^{cat}Ab on the surface will increase owing to the antibody-antigen binding interaction. Consequently, the homodimerization between the catenators will increase to form catenated antibodies in an arm-in-arm fashion (FIG. 1B and FIG. 1C). Importantly, the effective antigen-binding affinity of ^{cat}Ab will increase in parallel with the catenation, and the fold enhancement would depend on the degree of the catenation. In short, it appeared possible to enhance the antigen-binding avidity of the IgG-type antibodies by genetically fusing a weakly homodimer-forming protein.

2. Agent-Based Modeling (ABM) to Simulate the Behavior of ^{cat}Ab

Agent-based modeling (ABM) is a computational modeling approach that has been employed in a variety of research areas, including statistical physics and biological sciences. ABM enables the understanding of macroscopic behaviors of a complex system by defining a minimal set of rules governing microscopic behaviors of agents which compose the system.

We constructed an ABM to simulate the behavior of the ^{cat}Ab molecules on a target surface, where target antigen (Ag) molecules form antibody-binding sites. To circumvent complexity, we presumed that each binding site is a pair of two antigen molecules (2Ag) and ^{cat}Ab make a bivalent interaction with the binding site in a 1:1 stoichiometry to form an occupied binding site (^{cat}Ab-2Ag) (FIG. 3A, Left). For catenation to occur between two adjacent ^{cat}Ab-2Ag complexes, the distance between the centers of two adjacent complexes (d) should be closer than the reach length (L) defined as l+cl2, the sum of the linker length (l) and the half the catenator length (c) (FIG. 3A, Right). Therefore, multiple parameters affect the catenation on the target surface. In our ABM model, we regarded every possible binding site on the target surface as an individual agent in the ABM formalism, and each binding site is assigned to a fixed position on a three-dimensional (3D) surface with a periodic boundary condition. Three rules in our ABM govern the behaviors of the ^{cat}Ab molecules on the target surface. The first rule is about the intrinsic antibody-antigen binding. An unoccupied binding site binds to one free ^{cat}Ab through bivalent interaction to form an occupied binding site. Bound ^{cat}Ab may dissociate from the occupied binding site, leaving the binding site unoccupied. The equilibrium population of the occupied and unoccupied binding sites is determined by the antibody's intrinsic avidity for the antigen with no effect of the catenator on the antigen-binding avidity assumed. Then, the relative likelihood of the occupied state compared to the unoccupied state for any binding site (the likelihood of intrinsic antigen binding) is defined as [^{cat}Ab-2Ag]/[2Ag]) and thus can be expressed as [^{cat}Ab]/K_{D}, where [^{cat}Ab] is the concentration of ^{cat}Ab and K_{D} is the dissociation constant for the bivalent ^{cat}Ab-2Ag interaction (FIG. 3B, Left). The second rule is about catenation. A pair of ^{cat}Ab-2Ag complexes on the target surface can be bridged by intermolecular homodimerization between catenators (FIG. 3B, Middle). For a pair of ^{cat}Ab-2Ag complexes separated by d (FIG. 3A, Right), the relative likelihood of the catenation state to the non-catenation state is the ratio of the forward reaction rate (catenation) to the reverse reaction rate (decatenation). The forward reaction rate (R_{catenation}) and the reverse reaction rate (R_{decatenation}) are given as,

$${R}_{catenation} = {( {k}_{f} )}_{catination} * {( \frac{1}{{N}_{A}} * \frac{1}{{V}_{sphere}} )}^{2} * {V}_{overlap} ( d )$$

$${R}_{decatenation} = {( {k}_{r} )}_{catenation} * \frac{1}{{N}_{A}}$$

where k_{f} and kᵣ are the reaction rate constant of the forward and reverse reaction, respectively, N_{A} is the Avogadro number, Vₛₚₕₑᵣₑ is the local spherical volume within the reach of the catenator, and Vₒᵥₑᵣₗₐₚ(d) is the volume where two catenators can come in contact to form a homodimer (FIG. 3A, Right). In approximating the forward reaction rate, the catenator was assumed to sample Vₛₚₕₑᵣₑ uniformly. The relative likelihood, defined as R_{catenation}/R_{decatenation}, is then expressed as

$$\frac{{R}_{catenation}}{{R}_{decatenation}} = \frac{{( {k}_{f} )}_{catenator}}{{( {k}_{r} )}_{catenator}} * \frac{1}{{N}_{A}} * {( \frac{1}{{V}_{sphere}} )}^{2} * {V}_{overlap} ( d ) = \frac{f ( d )}{{( {K}_{D} )}_{catenator}}$$

$$where$$

$$f ( d ) = \frac{1}{{N}_{A}} * {( \frac{1}{{V}_{sphere}} )}^{2} * {V}_{overlap} ( d )$$

The relative likelihood is thus a function of d, and it is inversely proportional to the dissociation constant of the catenator in the bulk medium, (K_{D})_{catenator}. The function ƒ(d) can be viewed as the effective local concentration of the catenator in Vₒᵥₑᵣₗₐₚ(d). As expected, ƒ(d) and thus the relative likelihood is sensitively affected by the reach length and limited by the ^{cat}Ab-^{cat}Ab distance (FIG. 4A and FIG. 4B). Finally, the third rule is about restricted dissociation which assumes that catenated antibodies are not allowed to dissociate from the binding site, because the catenated arms would hold the dissociated antibody near its binding site, forcing it to rebind immediately. Under this assumption, antibody molecules are allowed to dissociate from the binding site, only if its catenator is not engaged in the homodimerization with nearby ^{cat}Ab-2Ag complexes (FIG. 3B, Right).

3. Simulations Show Enhancement of Antigen-Binding Avidity by Antibody Catenation

According to the postulated rules, we simulated the effects of the antibody catenation on the binding interaction between cat Ab and 2Ag on a three-dimensional surface by using the Markov Chain Monte-Carlo (MCMC) sampling method (Hooten M B & Wikle C K (2010) Statistical Agent-Based Models for Discrete Spatio-Temporal Systems. J Am Stat Assoc 105(489):236-248). Our sampling procedure is composed of three steps (FIG. 3C). The first step is an initialization, where a target surface with the antibody-binding sites is defined by specifying the coordinates for each site. A set of binding sites are positioned equidistant from each other or randomly positioned, and the inter-site distance or the number of binding sites were set as variables. The next step is an MCMC stochastic update step. In each update step, a binding site is randomly selected from the target surface, and the probability of changing the status of the selected binding site (occupied or not) is calculated by the Metropolis-Hasting algorithm (Hastings W K (1970) Monte-Carlo Sampling Methods Using Markov Chains and Their Applications. Biometrika 57(1):97-109; Grazzini J, Richiardi M G, & Tsionas M (2017) Bayesian estimation of agent-based models. J Econ Dyn Control 77:26-47). Then, the ‘on’ or ‘off’ state of this site is updated with the calculated probability. Accordingly, the catenation state is probabilistically updated for each update step. In the following sampling step, the total number of the occupied binding sites is counted, which is then collected through multiple simulation runs for the statistical analysis of the binding site occupancy and the effective antigen-binding avidity. The binding site occupancy is the mean value of the number of occupied binding sites collected for more than 1024 MCMC samplings. For each simulation, we calculated the mean binding occupancy and the effective dissociation constant, (K_{D})_{eff}, which takes into account the effect of the antibody catenation, is expressed as:

$${( {K}_{D} )}_{eff} = \frac{( 1 - Binding Site Occupancy ) * [ {}^{cat} Ab ]}{Binding Site Occupancy}$$

Since the catenator homodimerization should be affected by how the binding sites are distributed on a 3D surface, simulations were conducted for different arrays of binding sites. In the simulations, (K_{D})_{catenator} was the main variable, while other parameters were set constant. First, we simulated the binding sites forming a square lattice to find that the Ab-catenator exhibited enhanced binding site occupancy in a sigmodal manner, and that it could be enhanced to near full saturation by a catenator that forms a homodimer with quite low binding affinity. For instance, a ^{cat}Ab with (K_{D})_{catenator} of ˜1 μM exhibited ˜70-fold enhancement of the effective antigen-binding avidity (=reduction of (K_{D})_{eff}) in comparison with the same antibody without a fused catenator (FIG. 5). As a means of comparison across different simulation setups, we employed ‘(K_{=D})_{catenator,50}’ which is defined as the (K_{D})_{catenator} that enables half-maximal enhancement of the binding site occupancy (FIG. 5).

4. Comparison of the Simulations for Different Arrays of the Binding Sites

Next, we carried out simulations for regular arrays of the binding sites and for randomly distributed binding sites. Depending on the pattern of regularly distributed binding sites, the number of possible catenations for a given binding site (designated as connectivity number) varies: 3, 4 and 6 for a hexagonal, square or triangular array of the binding sites, respectively (FIG. 6A). Hence, the simulations assessed the influence of connectivity number on the catenation between cat Ab molecules on each surface. These three arrays showed varying but similar enhancement of the binding site occupancy and the effective antigen-binding avidity by the catenator (FIG. 6A). As expected, the higher the connectivity number was, the lower (K_{D})_{catenator} an array exhibited; the (K_{D})_{catenator,50} was 8.0, 9.2 and 12.2 μM for the hexagonal, square and triangular array of the binding sites, respectively. In addition, as the connectivity number increased, the effective antigen-binding avidity increased with the maximum 41-, 73- and 93-fold enhancement for triangular, square and hexagonal arrays, respectively (FIG. 6A). Thus, regardless of the distribution patterns, the effective antigen-binding avidity could be increased at least 41 folds in terms of (K_{D})_{eff} by catenator fusion to an antibody under the simulations conditions where the target surface contains 98 binding sites.

For the case of randomly distributed binding sites on a 3D surface, which is relevant to target antigen distribution on cell surfaces, we introduced the binding site density (p), the number of binding sites per unit area which is set to the square of the reach length (FIG. 6B). In the simulations, the total surface area was 5,760 nm², and the number of binding sites were 15, 30, 45, 90 or 120, which correspond to the p of 1.47, 2.94, 4.41, 8.82 or 11.76. Denser binding sites would increase the connectivity number for a given binding site. As expected, simulations with varying binding site densities showed that higher binding site density resulted in higher level of binding site saturation and more significant increase of the effective antigen-binding avidity; the maximum fold enhancement ranged from 15 (ρ=1.47) to 1,062 (ρ=11.76). Likewise, significant differences in the (K_{D})_{catenator,50} values were observed; e.g., the p of 1.47 required ˜18 times higher binding affinity between the catenators than the ρ of 11.76 to observe the half-maximal enhancement of the binding avidity, 4.2×10⁻⁶ M vs. 74×10⁻⁶ M in (K_{D})_{catenator,50} (FIG. 6B). The maximal saturation and onset (K_{D})_{catenator}, which begins to exert the catenation effect, were also considerably different. Thus, the catenation effects are sensitively affected by the binding site density, in contrast with the all-or-none catenation effects observed for the regular arrays of the binding sites (FIG. 6B). In particular, this enhancement was remarkably and sensitively affected by the (K_{D})_{catenator} values at high binding site density (ρ>4.41) (FIG. 6B). An even greater enhancement was observed upon further increasing the density of randomly distributed binding sites. Much greater enhancement was observed as we further increased the density of randomly distributed binding sites: ˜29,000 maximum fold enhancement at the p of 58.8 (FIG. 7), which roughly corresponds to two hundredths of the density of the HER2 receptor on HER2-overexpressing breast cancer cells (Peckys et al., (2019) Visualisation of HER2 homodimers in single cells from HER2 overexpressing primary formalin fixed paraffin embedded tumour tissue. Mol Med 25(1)). Together, the simulations show that randomly distributed binding sites at high density enormously enhance the effective antigen-binding avidity of cat Ab.

Additionally, we performed simulations for different values of [^{cat}Ab]/K_{D} to estimate the effect of K_{D} with respect to [^{cat}Ab]. Varying [^{cat}Ab]/K_{D} from 0.01 to 1.0 resulted in 85- to 900-fold enhancement of the antigen binding avidity, suggesting that the catenation effect works for a broad range of K_{D} values (FIG. 8A and FIG. 8B).

5. Proof-of-Concept Experiments

To experimentally test our concept, we chose SDF-1α(8-67), Sly1(254-316), eGFP (1-238) and HomoCC as a catenator. These are proteins that are small in size (molecular weight less than 30 kDa) and form homodimers with low affinity (K_{D}=0.1 μM or more), indicating that the protein fused to an antibody by a ˜40 Å-long linker would not form an intramolecular homodimer within a fusion protein. To find out how much the antigen-binding avidity of an antibody to the antigen is improved by fusion with the catenator, that is, to measure and compare the K_{D} value of the antibody itself to the antigen and the substantial dissociation constant ((K_{D})eff) value of ^{cat}Ab, biolayer interferometry (BLI) was performed. Seven antibodies Trastuzumab(N30A/H91A), Trastuzumab(N30A/H91A)/HetFc, Trastuzumab(N30A/H91A/Y100A), Trastuzumab(N30A/H91A/Y100A)/HetFc, glCV30, glCV30/HetFc, Obinutuzumab(F101L), Obinutuzumab(F101L)/HetFc) and three catenators (SDF-1α(8-67), SLy1(254-316), HomoCC) were analyzed, and two fusion positions of the catenators (heavy chain C-terminus or light chain C-terminus) were analyzed. The analysis method is to immobilize the target antigen on a biosensor tip, react with an antibody or antibody-catenator, obtain a kinetics curve of the process of association and dissociation between the two, and obtain a binding avidity (dissociation constant) therefrom.

5-1. When SDF-1α(8-67) is Fused to the C-Terminus of the Heavy Chain of Trastuzumab(N30A/H91A) or gICV30 (FIG. 9A and FIG. 9B)

SDF-1α(8-67) was fused by a 10-residue connecting linker (GGGGSGGGGS) to two different antibodies: glCV30, an antibody against the receptor-binding domain (RBD) of the severe acute respiratory syndrome coronavirus 2 (SARS-CoV-2) spike protein, and Trastuzumab, an antibody against HER2 protein. As Trastuzumab has N30A and H91A mutations in its light chain, its Fab fragment binds to the ectodomain of HER2 with the K_{D} of 353 nM (Slaga D, et α1. (2018) Avidity-based binding to HER2 results in selective killing of HER2-overexpressing cells by anti-HER2/CD3. Sci Transl Med 10(463)). glCV30 binds to the RBD with a similar binding affinity (K_{D}=407 nM) (Hurlburt N K, et α1. (2020) Structural basis for potent neutralization of SARS-CoV-2 and role of antibody affinity maturation. Nat Commun 11(1):5413). We produced the SDF-1α(8-67)-fused antibodies, Trastuzumab(N30A/H91A)-SDF-1α(8-67) and glCV-SDF-1α(8-67), and also the unmodified mother antibodies to compared their binding avidities by bio-layer interferometry (BLI) where respective target antigen was immobilized on a sensor tip. The mother antibodies, Trastuzumab (N30A/H91A) and glCV30 exhibited the K_{D} of 2.1 nM and 1.3 nM, respectively (FIG. 9A and FIG. 9B). The SDF-1α(8-67)-fused antibodies exhibited association kinetics similar to those of the mother antibodies; association rate constants (kₐ s) of the mother antibody Trastuzumab(N30A/H91A) and Trastuzumab(N30A/H91A)-SDF-1α(8-67) were 1.9×10⁵ Ms⁻¹ and 2.9×10⁵ Ms⁻¹, respectively. Similarly, kₐ s of glCV30 and glCV30-SDF-1α(8-67)^{(H)} were 6.7×10⁵ Ms⁻¹ and 7.7×10 ⁵ Ms⁻¹, respectively. However, the SDF-1α(8-67)-fused antibodies exhibited significantly different dissociation kinetics; dissociation rate constant (k_{d}s) of the mother antibody Trastuzumab(N30A/H91A) was 2.2×10⁻⁴ Ms⁻¹, whereas that of Trastuzumab(N30A/H91A)-SDF-1α(8-67) was <1.0×10⁻⁷ Ms⁻¹. Further, k_{d}s of the mother antibody glCV30 was 9.1×10⁻⁵ Ms⁻¹, whereas that of glCV30-SDF-1α(8-67)^{(H)} was <1.0×10⁻⁷ Ms⁻¹ (FIG. 9A and FIG. 9B). These observed kinetics are consistent with the expectation that fused SDF-1α would not affect the association of the antibodies, but would slow down the dissociation of the SDF-1α-fused antibodies into the bulk solution due to the catenation. As a result, Trastuzumab(N30A/H91A)-SDF-1α(8-67) exhibited the K_{D} of <0.01 nM, at least 110-fold higher binding avidity compared with the mother antibody Trastuzumab(N30A/H91A), and likewise, the SDF-1α(8-67) fusion to glCV30 increased the binding avidity by at least 130 folds, demonstrating that one-digit nanomolar binding avidity of an antibody can be increased to picomolar binding avidity by fusing a weakly homodimerizing protein.

5-2. When SDF-1α(8-67) is Fused to the C-Terminus of the Light Chain of Trastuzumab(N30A/H91A) (FIG. 10A)

Even in this case, dissociation of Trastuzumab(N30A/H91A)-SDF-1 α(8-67)^{(L)} was not observed in the dissociation phase, and therefore the binding avidity ((K_{D})_{eff}<0.01 nM) increased at least 210-fold compared to that of Trastuzumab (N30A/H91A) (K_{D}=2.1 nM) (FIG. 10A).

5-3. When HomoCC is Fused to the C-Terminus of the Heavy Chain of Trastuzumab(N30A/H91A) (FIG. 10B)

Even in this case, the binding avidity of Trastuzumab(N30A/H91A)-HomoCC (H) in the dissociation phase ((K_{D})_{eff}<0.01 nM) increased at least 210-fold compared to that of Trastuzumab (N30A/H91A) (K_{D}=2.1 nM) (FIG. 10B).

5-4. When SDF-1α(8-67) and SLy1(254-316) are Fused to the C-Terminus of the Heavy Chain of Trastuzumab(N30A/H91A/Y100A) Containing Heterodimeric Fc (FIG. 11A to FIG. 11C)

Wild-type Fc forms a homodimer, but attempts have been made to generate a heterodimeric Fc to make a bispecific antibody. It has been reported that a KH/KH⁻¹ pair, which a knobs-into-holes type mutations (knob: T366W; hole: T366S, L358V, Y407A) are introduced in the heavy chain C_{H3} domain, forms a heterodimer well (Leaver-Fay et al., (2016). Computationally Designed Bispecific Antibodies using Negative State Repertoires Structure. 24, 641-651). The present inventors prepared an antibody (Trastuzumab (N30A/H91A/Y100A)/HetFc) in which a Fab of Trastuzumab (N30A/H91A/Y100A) was attached to the heterodimeric Fc, and antibody-catenators (SLy1(254-316)^{(H)}-Trastuzumab(N30A/H91A/Y100A)/HetFc-SDF-1α(8-67)^{(H)}) in which SLy1(254-316) was fused to KH Fc and SDF-1α (8-67) was fused to KH⁻¹-1 Fc, respectively. In addition, for a control experiment, an antibody-catenator (SLy1(254-316)^{(H)}-Trastuzumab(N30A/H91A/Y100A)/HetFc) in which only SLy1(254-316) was fused was also prepared. Further, mScarlet was tagged to the C-terminus of the light chain of both antibodies. Both antibodies appeared as monomers in solution and were readily isolated (FIG. 11A). Fusion of two different catenators results in catenation on the target surface, whereas fusion of one catenator cannot result in catenation (FIG. 11B). The binding avidity of Trastuzumab(N30A/H91A/Y100A) to HER2 is very weak(K_{D}=243 nM), but the binding avidity of SLy1(254-316)^{(H)}-Trastuzumab(N30A/H91A/Y100A)/HetFc-SDF-1α(8-67)^{(H)} increased by about 304 folds ((K_{D})_{eff}<0.8 nM) (FIG. 11C). This increase of antigen-binding avidity is due to the catenation effect. This is because when only SLy1 (254-316) is fused (i.e., there is only one catenator arm), the increase in bonding strength is not observed (FIG. 11C). This result shows that the antibody in the form of a bispecific antibody can also amplify its binding avidity to an antigen by fusing a catenator.

Summarizing the results, antibody catenation is a method capable of increasing the antigen-binding avidity of an antibody to an antigen hundreds to thousands of times regardless of the type of antibody and regardless of the fusion site of the catenator. In addition, since the improvement of the antigen-binding avidity was shown to be independent of the type of catenator, it can be concluded that this method is not limited to a specific IgG antibody and is applicable to all IgG antibodies.

5-5. When SLy1(254-316) and SDF-1α(8-67) are Fused to the C-Terminus of Heterodimeric Fc of Obinutuzumab(F101L) (FIG. 12A)

An anti-lymphoma antibody, Obinutuzumab exhibits strong avidity (K_{D}=0.097 nM) for its target antigen, CD20 (Kumar et al., (2020). Binding mechanisms of therapeutic antibodies to human CD20. Science 369, 793-799; Mossner et al., (2010). Increasing the efficacy of CD20 antibody therapy through the engineering of a new type II anti-CD20 antibody with enhanced direct and immune effector cell-mediated B-cell cytotoxicity. Blood 115, 4393-4402). Obinutuzumab(Y101L)/HetFc was prepared using a heterodimeric Fc and Obinutuzumab (Y101L), in which a mutation was introduced to artificially lower antigen binding ability, and then SLy1(254-316)-Obinutuzumab(Y101L)/HetFc-SDF-1α(8-67)^{(H)} was prepared using the same. The binding avidity of Obinutuzumab(Y101L) to CD20 is weak (K_{D}=30.4 nM), but the binding avidity of SLy1(254-316)^{(H)}-Obinutuzumab(Y101L)/HetFc-SDF-1α(8-67)^{(H)} increased by about 234 folds ((K_{D})_{eff}=0.13 nM) (FIG. 12A).

6. Amplification of Antibody-Catenator Binding Avidity in Cancer Cells

Characteristics of catenated antibodies were analyzed using cancer cells. The binding avidity of the antibody-catenator to the antigen increases in proportion to the density of the antigen. In general, anticancer target molecules located on the cell surface are expressed relatively more in cancer cells than in normal cells, and thus, antibody-catenator will bind better to the surface of cancer cells with a high density of antigens than to the surface of normal cells with a low number or density of antigens. To demonstrate this, experiments were performed on a breast cancer cell line that grows by attaching to the surface and a blood cancer cell line that grows in a suspended state.

6-1. Enhanced Binding Avidity of Catenated Antibodies to a Hematological Cancer Cell Line (FIG. 12B)

Since a lymphoma cell line, SU-DHL-5 (DSMZ) is a floating cell, the cell binding activity of the antibody was analyzed by fluorescence assisted cell sorting (FACS) method instead of confocal microscopy. Obinutuzumab (F101L) antibody shows weak binding to SU-DHL5 cells due to its low binding avidity to CD20, whereas 90% of SLy1(254-316)^{(H)}-Obinutuzumab(F101L)/HetFc-SDF-1α(8-67)^{(H)} antibody bound to SU-DHL5 cells even at a low concentration (10 nM) (FIG. 12B).

6-2. Enhanced Binding Avidity of Catenated Antibodies to Breast Cancer Cell Lines (FIG. 13)

As a result of treatment with Trastuzumab(N30A/H91A) and Trastuzumab(N30A/H91A)-SDF-1α(8-67)^{(H)} to 4 types of breast cancer cell lines with different expression of the target antigen HER2, Trastuzumab(N30A/H91A)-SDF-1α(8-67)^{(H)} showed much higher binding avidity compared to Trastuzumab(N30A/H91A), and the binding degree increased in proportion to the level of HER2 expression (FIG. 13).

6-3. Selective Binding Avidity to Breast Cancer Cell Lines by Antibody Catenation (FIG. 14)

When Trastuzumab(N30A/H91A) was treated to two breast cancer cell lines MCF-7 and BT-474, which have different expression of the target antigen HER2, as expected, the Trastuzumab (N30A/H91A) antibody binds better to BT-474, which has a higher level of HER2 expression, than MCF-7 (FIG. 14, top left). Treatment with Trastuzumab(N30A/H91A)-SDF-1α(8-67)^{(H)} at the same concentration showed that this catenated antibody bound much more strongly and rapidly to BT-474 cells than the non-catenated antibody. However, the binding could not be observed in MCF-7 cells (FIG. 14). Thus, the catenated antibody has a property of selectively binding to cells having a high antigen density even though it can simultaneously access cells having different target antigen densities. This result suggests that the antibody-catenator has the ability to selectively bind to cancer cells rather than normal cells.

7. Increased Virus Neutralizing Activity of Antibody-Catenators (FIG. 15)

The SARS-CoV-2 neutralizing activity of CV30, glCV30 and glCV30-SDF-1α(8-67) was compared using VSV pseudotyped with the SARS-CoV-2 spike protein. The virus has multiple copies of the spike protein on its envelope to which glCV30-SDF-1α (8-67) molecules can be catenated. Each of the three antibodies (CV30, glCV30 and glCV30-SDF-1α(8-67)) was serially diluted and added to rVSV-ΔG-Luc containing the SARS-CoV-2 spike protein of Wuhan-Hu-1 strain. The mixture was incubated with HEK293T-hACE2 cells and luciferase activity was measured.

As shown in FIG. 15, CV30, glCV30, and glCV30-SDF-1α had IC₅₀ values of 0.25, 3.22, and 0.21 μg/ml, respectively. In particular, compared to glCV30 (IC₅₀ of 3.22 μg/ml), glCV30-SDF-1α(8-67) (IC₅₀ of 0.21 μg/ml) showed ˜15-fold lower IC₅₀ values. In addition, this neutralizing potency of glCV30-SDF-1α(8-67) (IC₅₀ of 0.21 μg/ml) was similar to that of CV30 (IC₅₀ of 0.25 μg/ml), which increased the antigen-binding avidity of glCV30. The dissociation constant of CV30, as measured by BLI, is 0.01 nM or less, similar to that of glCV30-SDF-1α. These results demonstrate that an antibody with weak antigen binding avidity can be converted into a strong antibody by fusing a catenator.

8. Effector Functions of Antibody-Catenators (FIG. 16)

SLy1(254-316)-Obinutuzumab(Y101L)/HetFc-SDF-1α(8-67)^{(H)}, in which two catenators were fused, was prepared using the parental antibody Obinutuzumab (Y101L) and HetFc, and the original Obinutuzumab was also prepared. Effector functions for each of them were analyzed.

Antibody-dependent cytotoxicity (ADCC) and complement-dependent cytotoxicity (CDC) of Obinutuzumab, Obinutuzumab(Y101L), SLy1(254-316)^{(H)}-Obinutuzumab(Y101L)/HetFc-SDF-1α(8-67)^{(H)} were measured using the Incucyte® Live-Cell Analysis System. To measure ADCC, peripheral blood mononuclear cells (PBMC) extracted from the blood of two donors (marked as Donor) were used respectively (Effector cell:Target cell ratio=10:1).

As a result, SLy1(254-316)^{(H)}-Obinutuzumab (Y101L)/HetFc-SDF-1α(8-67)^{(H)} showed a superior ADCC efficacy than Obinutuzumab (Y101L), and the degree was similar to that of the original Obinutuzumab(K_{D}=0.097 nM). For the measurement of CDC, only human serum was treated and measured, and three antibodies did not show significant CDC.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are intended to be embraced by the claims

What is claimed is:

1. A conjugate comprising (i) an antibody or fragment thereof, and (ii)catenator polypeptides fused to each of the two heavy chain C-termini or to each of the two light chain C-termini of the antibody or fragment thereof, wherein the catenator polypeptide satisfies one or more of the following criteria:
(a) is capable of forming a dimer;
(b) the dissociation constant (K_{D}) of dimer formation is between 0.1 μM and 500 μM;
(c) a molecular weight is between 3 kDa and 30 kDa; and
(d) On the surface where target antigens to which the antibody specifically binds are present, pairs of the conjugate-antigen complexes adjacent to each other are catenated by intermolecular dimerization between catenator polypeptides.

2. The conjugate of claim 1, wherein the catenator polypeptide is fused to the antibody or fragment thereof through a linker.

3. The conjugate of claim 1, wherein the catenator polypeptide is a polypeptide of SEQ ID NO: 1, SEQ ID NO: 2 or SEQ ID NO: 3.

4. The conjugate of claim 1, wherein the antibody is an IgG type antibody.

5. The conjugate of claim 1, wherein the antibody is a therapeutic antibody, a diagnostic antibody, a monoclonal antibody, a chimeric antibody, a humanized antibody, or a human antibody.

6. The conjugate of claim 1, wherein the antibody is an antibody of an antibody-drug conjugate (ADC).

7. The conjugate of claim 1, wherein the antibody is a multispecific antibody.

8. The conjugate of claim 1, wherein the fragment of antibody is Fc or F(ab′)₂.

9. The conjugate of claim 8, wherein the fragment of antibody is an Fc fragment of a drug-Fc fragment conjugate.

10. The conjugate of claim 1, wherein the antibody is a secondary antibody of a sandwich immunoassay.

11. A composition for enhancing antigen-binding avidity of an antibody, the composition comprising the conjugate of claim 1, which is characterized in that, on the surface where target antigens to which the antibody included in the conjugate specifically binds are present, pairs of the conjugate-antigen complexes adjacent to each other are catenated by intermolecular dimerization between catenator polypeptides, thereby suppressing dissociation of the antibody.

12. A method for enhancing antigen-binding avidity of an antibody, comprising treating the conjugate of claim 1 on the surface where target antigens to which the antibody specifically binds are present, which is characterized in that, on the surface, pairs of the conjugate-antigen complexes adjacent to each other are catenated by intermolecular dimerization between catenator polypeptides, thereby suppressing dissociation of the antibody.


SEQUENCE LISTING (3 sequences)

SEQ ID NO: 1 - protein, 60 aa, synthetic construct
  source 1..60: note = SDF-1 alpha (8-67)
RCPCRFFESHVARANVKHLKILNTPACALQIVARLKNNNEQVCIDPKLKWIQEYLEKALN

SEQ ID NO: 2 - protein, 63 aa, synthetic construct
  source 1..63: note = SLy1 (254-316)
KTLHELLERIGLEEHTSTLLLNGYQTLEDFKELRETHLNELNIMDPQHRAKLLTAAELLLDYD

SEQ ID NO: 3 - protein, 71 aa, synthetic construct
  source 1..71: note = HomoCC
DEEQRKVVEEDLKVLEHLRRVVERKEHLVRDAYEETFDDQQREVVREKLKVLEHLEKVIERDRHLSSRPGL